SEQUENCE LISTING (2 sequences)

SEQ ID NO: 1 - protein, 609 aa, Homo sapiens [sequence omitted]

SEQ ID NO: 2 - protein, 224 aa, Homo sapiens
MEKLLCFLVLTSLSHAFGQTDMSRKAFVFPKESDTSYVSLKAPLTKPLKAFTVCLHFYTELSSTRGYSIFSYATKRQDNEILIFWSKDIGYSFTVGGSEILFEVPEVTVAPVHICTSWESASGIVEFWVDGKPRVRKSLKKGYTVGAEASIILGQEQDSFGGNFEGSQSLVGDIGNVNMWDFVLSPDEINTIYLGGPFSPNVLNWRALKYEVQGEVFTKPQLWP

(19) United States
(12) Patent Application Publication
(10) Pub. No.: US 2022/0146501 A1
(43) Pub. Date: May 12, 2022

(54) ASSAY METHODS FOR THE DETECTION OF HUMAN SERUM ALBUMIN, VITAMIN D, C-REACTIVE PROTEIN, AND ANTI-TRANSGLUTAMINASE AUTOANTIBODY

(71) Applicant: PROCISEDX INC., San Diego, CA (US)

(72) Inventors: JINYAO ZHOU, SAN DIEGO, CA (US); RAJ SRIKRISHNAN, SR., SAN DIEGO, CA (US); MICHAEL HALE, SAN DIEGO, CA (US); STEFAN WESTIN, SAN DIEGO, CA (US); MARK RENSHAW, SAN DIEGO, CA (US); LARRY MIMMS, SAN DIEGO, CA (US); RUKMINI REDDY, SAN DIEGO, CA (US)

(73) Assignee: PROCISEDX INC., San Diego, CA (US)

(21) Appl. No.: 17/503,942

(22) Filed: Oct. 18, 2021

Related U.S. Application Data
(63) Continuation of application No. PCT/US2020/032965, filed on May 14, 2020.
(60) Provisional application No. 62/852,174, filed on May 23, 2019.
(60) Provisional application No. 62/857,134, filed on Jun. 4, 2019.
(60) Provisional application No. 62/863,120, filed on Jun. 18, 2019.
(60) Provisional application No. 62/866,506, filed on Jun. 25, 2019.

(51) Int. Cl. G01N 33/542; G01N 33/82

(52) U.S. Cl. CPC G01N 33/542; G01N 33/82; G01N 2333/765; G01N 2333/4737; G01N 2333/91085

(57) ABSTRACT

An assay method for detecting the presence or amount of human serum albumin vitamin D, C-reactive protein, or anti-transglutaminase autoantibody (ATA) immunoglobulin A (IgA) and/or ATA IgG in a sample using fluorescence resonance energy transfer (FRET).

### CROSS-REFERENCES TO RELATED APPLICATIONS

This application is a continuation of PCT/US2020/032965, filed May 14, 2020, which claims priority to U.S. Provisional Application No. 62/852,174, filed May 23, 2019, U.S. Provisional Application No. 62/857,134, filed Jun. 4, 2019, U.S. Provisional Application No. 62/863,120, filed Jun. 18, 2019, and U.S. Provisional Application No. 62/866,506, filed Jun. 25, 2019, the disclosures of which are hereby incorporated by reference in their entirety for all purposes.

### SEQUENCE LISTING

The instant application contains a Sequence Listing which has been submitted electronically in ASCII format and is hereby incorporated by reference in its entirety. Said ASCII copy, created on Jan. 6, 2022, is named 105741-029210US-1267836 SL.txt and is 7,745 bytes in size.

### BACKGROUND OF THE INVENTION

Some biological assays rely on time-resolved fluorescence resonance energy transfer (TR-FRET) mechanisms where two fluorophores are used. Biological materials are typically prone to autofluorescence, which can be minimized by utilizing time-resolved FRET. TR-FRET takes advantage of rare earth elements such as lanthanides (e.g., europium and terbium), which have exceptionally long fluorescence emission half-lives. In these assays, energy is transferred between a donor fluorophore and an acceptor fluorophore if the two fluorophore are in close proximity to one another. Excitation of the donor (e.g., cryptate) by an energy source (e.g., UV light) produces an energy transfer to the acceptor, if the two fluorophores are within a given proximity. In turn, the acceptor emits light at its characteristic wavelength. In order for TR-FRET to occur, the fluorescence emission spectrum of the donor molecule must overlap with the absorption or excitation spectrum of the acceptor chromophore. Moreover, the fluorescence lifetime of the donor molecule must be of sufficient duration to allow TR-FRET to occur.

Cryptates can be used in various bioassays formats. Cryptates are complexes that include a macrocycle within which a lanthanide ion such as terbium or europium is tightly embedded or chelated. This cage like structure is useful for collecting irradiated energy and transferring the collected energy to the lanthanide ion. The lanthanide ion can release the energy with a characteristic fluorescence.

U.S. Pat. No. 6,406,297 is titled “Salicylamide-lanthanide complexes for use as luminescent markers.” This patent is directed to luminescent lanthanide metal chelates comprising a metal ion of the lanthanide series and a complexing agent comprising a salicylamidyl moiety. This patent is hereby incorporated by reference.

U.S. Pat. No. 6,515,113 is titled “Phthalamide lanthanide complexes for use as luminescent markers.” This patent is directed to luminescent lanthanide metal chelates comprising a metal ion of the lanthanide series and a complexing agent comprising a phthalamidyl moiety. This patent is hereby incorporated by reference.

WO2015157057 is titled “Macrocycles” and relates to chemical compounds and complexes that can be used in therapeutic and diagnostic applications. This publication contains cryptate molecules useful for labeling biomolecules. This publication is hereby incorporated by reference.

WO2018130988 discloses cryptates derivatives and conjugates thereof with excellent fluorescent properties. The cryptates are useful in biological assays and methods for the detection and identification of various analytes.

In view of the foregoing, what is needed in the art is a homogeneous assay that can measure the presence or amount of a biomolecule to provide an increase in flexibility, reliability, and sensitivity in addition to higher throughput. The present disclosure provides this and other needs.

### BRIEF SUMMARY

In one aspect, the present disclosure provides an assay method for detecting the presence or amount of human serum albumin in a sample, the method comprising:

contacting the sample with a complex comprising an anti-human serum albumin antibody labeled with a donor fluorophore and an isolated human serum albumin labeled with an acceptor fluorophore, wherein the complex emits a fluorescence emission signal associated with fluorescence resonance energy transfer (FRET) when the donor fluorophore is excited using a light source;

incubating the sample with the complex for a time sufficient for human serum albumin in the sample to compete for binding to the anti-human serum albumin antibody labeled with the donor fluorophore; and

exciting the sample using a light source to detect the fluorescence emission signal associated with FRET,

wherein an absence of the fluorescence emission signal or a decrease in the fluorescence emission signal relative to the fluorescence emission signal initially emitted by the complex indicates the presence or amount of human serum albumin in the sample.

In another aspect, the disclosure provides an assay method for detecting the presence or amount of human serum albumin in a sample, the method comprising:

contacting the sample with a complex comprising an anti-human serum albumin antibody labeled with an acceptor fluorophore and an isolated human serum albumin labeled with an donor fluorophore, wherein the complex emits a fluorescence emission signal associated with fluorescence resonance energy transfer (FRET) when the donor fluorophore is excited using a light source;

incubating the sample with the complex for a time sufficient for human serum albumin in the sample to compete for binding to the anti-human serum albumin antibody labeled with the acceptor fluorophore; and

exciting the sample using a light source to detect a fluorescence emission signal associated with FRET,

wherein an absence of the fluorescence emission signal or a decrease in the fluorescence emission signal relative to the fluorescence emission signal initially emitted by the complex indicates the presence or amount of human serum albumin in the sample.

In some embodiments, the concentration of human serum albumin in the blood is about 3 g/L to about 500 g/L. In some embodiments, the normal concentration of human serum albumin in the blood is about 35 g/L to about 50 g/L. In some embodiments, an elevated concentration of human serum albumin in the blood is at least 50 g/L. In some embodiments, an elevated concentration of human serum albumin in the blood is at least 100 g/L. In some embodiments, a low concentration of human serum albumin in the blood is below 35 g/L. In some embodiments, a low concentration of human serum albumin in the blood is below 20 g/L.

In another aspect, the present disclosure provides an assay method for detecting the presence or amount of vitamin D in a sample, the method comprising:

contacting the sample with a complex comprising a vitamin D-binding agent labeled with a donor fluorophore and an isolated vitamin D labeled with an acceptor fluorophore, wherein the complex emits a fluorescence emission signal associated with fluorescence resonance energy transfer (FRET) when the donor fluorophore is excited using a light source;

incubating the sample with the complex for a time sufficient for vitamin D in the sample to compete for binding to the vitamin D-binding agent labeled with an donor fluorophore; and

exciting the sample using a light source to detect a fluorescence emission signal associated with FRET,
wherein an absence of the fluorescence emission signal or a decrease in the fluorescence emission signal relative to the fluorescence emission signal initially emitted by the complex indicates the presence or amount of vitamin D in the sample.

In another aspect, the present disclosure provides an assay method for detecting the presence or amount of vitamin D in a sample, the method comprising:

contacting the sample with a complex comprising a vitamin D-binding agent labeled with an acceptor fluorophore and an isolated vitamin D labeled with an donor fluorophore, wherein the complex emits a fluorescence emission signal associated with fluorescence resonance energy transfer (FRET) when the donor fluorophore is excited using a light source;

incubating the sample with the complex for a time sufficient for vitamin D in the sample to compete for binding to the vitamin D-binding agent labeled with an acceptor fluorophore; and

exciting the sample using a light source to detect a fluorescence emission signal associated with FRET,
wherein an absence of the fluorescence emission signal or a decrease in the fluorescence emission signal relative to the fluorescence emission signal initially emitted by the complex indicates the presence or amount of vitamin D in the sample.

In some embodiments, the concentration of vitamin D in the blood is about 2 ng/mL to about 500 ng/mL (e.g., about 2 ng/mL, 5 ng/mL, 10 ng/mL, 20 ng/mL, 30 ng/mL, 40 ng/mL, 50 ng/mL, 60 ng/mL, 70 ng/mL, 80 ng/mL, 90 ng/mL, 100 ng/mL, 110 ng/mL, 120 ng/mL, 130 ng/mL, 140 ng/mL, 150 ng/mL, 160 ng/mL, 170 ng/mL, 180 ng/mL, 190 ng/mL, 200 ng/mL, 210 ng/mL, 220 ng/mL, 230 ng/mL, 240 ng/mL, 250 ng/mL, 260 ng/mL, 270 ng/mL, 280 ng/mL, 290 ng/mL, 300 ng/mL, 310 ng/mL, 320 ng/mL, 330 ng/mL, 340 ng/mL, 350 ng/mL, 360 ng/mL, 370 ng/mL, 380 ng/mL, 390 ng/mL, 400 ng/mL, 410 ng/mL, 420 ng/mL, 430 ng/mL, 440 ng/mL, 450 ng/mL, 460 ng/mL, 470 ng/mL, 480 ng/mL, 490 ng/mL, or 500 ng/mL).

In some embodiments, the normal concentration of vitamin D in the blood is about 20 ng/mL to about 50 ng/mL (e.g., about 20 ng/mL, 23 ng/mL, 25 ng/mL, 27 ng/mL, 29 ng/mL, 31 ng/mL, 33 ng/mL, 35 ng/mL, 37 ng/mL, 39 ng/mL, 41 ng/mL, 43 ng/mL, 45 ng/mL, 47 ng/mL, 49 ng/mL, or 50 ng/mL).

In some embodiments, an elevated concentration of vitamin D in the blood is at least 50 ng/mL (e.g., 60 ng/mL, 70 ng/mL, 80 ng/mL, 90 ng/mL, 100 ng/mL, 110 ng/mL, 120 ng/mL, 130 ng/mL, 140 ng/mL, 150 ng/mL, 160 ng/mL, 170 ng/mL, 180 ng/mL, 190 ng/mL, 200 ng/mL, 210 ng/mL, 220 ng/mL, 230 ng/mL, 240 ng/mL, 250 ng/mL, 260 ng/mL, 270 ng/mL, 280 ng/mL, 290 ng/mL, 300 ng/mL, 310 ng/mL, 320 ng/mL, 330 ng/mL, 340 ng/mL, 350 ng/mL, 360 ng/mL, 370 ng/mL, 380 ng/mL, 390 ng/mL, 400 ng/mL, 410 ng/mL, 420 ng/mL, 430 ng/mL, 440 ng/mL, 450 ng/mL, 460 ng/mL, 470 ng/mL, 480 ng/mL, 490 ng/mL, or 500 ng/mL).

In some embodiments, an elevated concentration of vitamin D in the blood is at least 100 ng/mL (e.g., at least 110 ng/mL, 120 ng/mL, 130 ng/mL, 140 ng/mL, 150 ng/mL, 160 ng/mL, 170 ng/mL, 180 ng/mL, 190 ng/mL, 200 ng/mL, 210 ng/mL, 220 ng/mL, 230 ng/mL, 240 ng/mL, 250 ng/mL, 260 ng/mL, 270 ng/mL, 280 ng/mL, 290 ng/mL, 300 ng/mL, 310 ng/mL, 320 ng/mL, 330 ng/mL, 340 ng/mL, 350 ng/mL, 360 ng/mL, 370 ng/mL, 380 ng/mL, 390 ng/mL, 400 ng/mL, 410 ng/mL, 420 ng/mL, 430 ng/mL, 440 ng/mL, 450 ng/mL, 460 ng/mL, 470 ng/mL, 480 ng/mL, 490 ng/mL, or 500 ng/mL).

In some embodiments, a low concentration of vitamin D in the blood is below 20 ng/mL (e.g., 18 ng/mL, 16 ng/mL, 14 ng/mL, 12 ng/mL, 10 ng/mL, 8 ng/mL, 6 ng/mL, or 4 ng/mL).

In some embodiments, a low concentration of vitamin D in the blood is below 10 ng/mL (e.g., 8 ng/mL, 6 ng/mL, or 4 ng/mL).

In another aspect, the present disclosure provides an assay method for detecting the presence or amount of C-reactive protein (CRP) in a sample, the method comprising:

contacting the sample with a complex comprising an anti-C-reactive protein antibody labeled with a donor fluorophore and an isolated C-reactive protein labeled with an acceptor fluorophore, wherein the complex emits a fluorescence emission signal associated with fluorescence resonance energy transfer (FRET) when the donor fluorophore is excited using a light source;

incubating the sample with the complex for a time sufficient for C-reactive protein in the sample to compete for binding to the anti-C-reactive protein antibody labeled with the donor fluorophore; and

exciting the sample using a light source to detect a fluorescence emission signal associated with FRET,

wherein an absence of the fluorescence emission signal or a decrease in the fluorescence emission signal relative to the fluorescence emission signal initially emitted by the complex indicates the presence or amount of C-reactive protein in the sample.

In another aspect, the present disclosure provides an assay method for detecting the presence or amount of C-reactive protein in a sample, the method comprising:

contacting the sample with a complex comprising an anti-C-reactive protein antibody labeled with an acceptor fluorophore and an isolated C-reactive protein labeled with a donor fluorophore, wherein the complex emits a fluorescence emission signal associated with fluorescence resonance energy transfer (FRET) when the donor fluorophore is excited using a light source;

incubating the sample with the complex for a time sufficient for C-reactive protein in the sample to compete for binding to the anti-C-reactive protein antibody labeled with the acceptor fluorophore; and

exciting the sample using a light source to detect a fluorescence emission signal associated with FRET,

wherein an absence of the fluorescence emission signal or a decrease in the fluorescence emission signal relative to the fluorescence emission signal initially emitted by the complex indicates the presence or amount of C-reactive protein in the sample.

In some embodiments, the normal concentration of C-reactive protein in the blood is below 3 mg/L. In some embodiments, an elevated concentration of C-reactive protein in the blood is at least 15 mg/L. In certain embodiments, an elevated concentration of C-reactive protein in the blood is at least 30 mg/L.

In another aspect, the present disclosure provides an assay method for detecting the presence or amount of anti-transglutaminase autoantibody (ATA) immunoglobulin A (IgA) and/or ATA immunoglobulin G (IgG) in a sample, the method comprising:

contacting the sample with a complex comprising an anti-tissue transglutaminase antibody labeled with a donor fluorophore and an isolated tissue transglutaminase labeled with an acceptor fluorophore, wherein the anti-tissue transglutaminase antibody comprises a binding epitope to tissue transglutaminase, and wherein the complex emits a fluorescence emission signal associated with fluorescence resonance energy transfer (FRET) when the donor fluorophore is excited using a light source;

incubating the sample with the complex for a time sufficient for ATA IgA and/or ATA IgG in the sample to compete for binding to the isolated tissue transglutaminase labeled with the acceptor fluorophore; and

exciting the sample using a light source to detect a fluorescence emission signal associated with FRET,

wherein an absence of the fluorescence emission signal or a decrease in the fluorescence emission signal relative to the fluorescence emission signal initially emitted by the complex indicates the presence or amount of ATA IgA and/or ATA IgG in the sample.

In another aspect, the present disclosure provides an assay method for detecting the presence or amount of anti-transglutaminase autoantibody (ATA) immunoglobulin A (IgA) and/or ATA immunoglobulin G (IgG) in a sample, the method comprising:

contacting the sample with a complex comprising an anti-tissue transglutaminase antibody labeled with an acceptor fluorophore and an isolated tissue transglutaminase labeled with a donor fluorophore, wherein the anti-tissue transglutaminase antibody comprises a binding epitope to tissue transglutaminase, and wherein the complex emits a fluorescence emission signal associated with fluorescence resonance energy transfer (FRET) when the donor fluorophore is excited using a light source;

incubating the sample with the complex for a time sufficient for ATA IgA and/or ATA IgG in the sample to compete for binding to the isolated tissue transglutaminase labeled with the donor fluorophore; and

exciting the sample using a light source to detect a fluorescence emission signal associated with FRET,

wherein an absence of the fluorescence emission signal or a decrease in the fluorescence emission signal relative to the fluorescence emission signal initially emitted by the complex indicates the presence or amount of ATA IgA and/or ATA IgG in the sample.

In another aspect of the disclosure, the disclosure provides an assay method that can detect, as well as distinguish, the presence or amount of anti-transglutaminase autoantibody (ATA) immunoglobulin A (IgA) and ATA immunoglobulin G (IgG) in a sample, the method comprising:

contacting the sample with a complex comprising an anti-tissue transglutaminase antibody labeled with a donor fluorophore (or a first acceptor fluorophore) and an isolated tissue transglutaminase labeled with a first acceptor fluorophore (or a donor fluorophore), wherein the anti-tissue transglutaminase antibody comprises a binding epitope to tissue transglutaminase, and wherein the complex emits a fluorescence emission signal associated with fluorescence resonance energy transfer (FRET) when the donor fluorophore is excited using a light source;

incubating the sample with the complex for a time sufficient for ATA IgA and/or ATA IgG in the sample to compete for binding to the isolated tissue transglutaminase labeled with the first acceptor fluorophore (or a donor fluorophore);

further incubating the sample with an anti-IgA antibody labeled with a second acceptor fluorophore and an anti-IgG antibody labeled with a third acceptor fluorophore; and

exciting the sample using a light source to detect fluorescence emission signals associated with FRET,

wherein a detection of a fluorescence signal emitted by the second acceptor fluorophore indicates the presence or amount of ATA IgA and a detection of a fluorescence signal emitted by the third acceptor fluorophore indicates the presence or amount of ATA IgG in the sample.

In some embodiments, the concentration of ATA IgA in the blood is about 7 mg/dL to about 4,000 mg/dL. In certain embodiments, the normal concentration of ATA IgA in the blood is about 70 mg/dL to about 400 mg/dL. In certain embodiments, an elevated concentration of ATA IgA in the blood is at least above 400 mg/dL. In particular embodiments, an elevated concentration of ATA IgA in the blood is at least above 800 mg/dL.

In some embodiments, the concentration of ATA IgG in the blood is about 20 mg/dL to about 4,000 mg/dL. In certain embodiments, the normal concentration of ATA IgG in the blood is about 200 mg/dL to about 400 mg/dL. In certain embodiments, an elevated concentration of ATA IgG in the blood is at least above 400 mg/dL. In particular embodiments, an elevated concentration of ATA IgG in the blood is at least above 800 mg/dL.

In some embodiments of the methods described herein, the FRET emission signals are time resolved FRET emission signals.

In some embodiments, the sample is a biological sample, such as whole blood, urine, a fecal specimen, plasma, or serum. In particular embodiments, the biological sample is whole blood.

In some embodiments, the donor fluorophore is a terbium cryptate. In some embodiments, the acceptor fluorophore is selected from the group consisting of fluorescein-like (green zone), Cy5, DY-647, Alexa Fluor 488, Alexa Fluor 546, Alexa Fluor 647, allophycocyanin (APC), and phycoerythrin (PE).

In some embodiments, the light source provides an excitation wavelength between about 300 nm to about 400 nm. In some embodiments, the fluorescence emission signals emit emission wavelengths that are between about 450 nm to about 700 nm.

These and other aspects, objects and embodiments will become more apparent when read with the detailed description and figures that follow.

### BRIEF DESCRIPTION OF THE DRAWINGS

FIG. 1 illustrates one embodiment of the present disclosure directed to human serum albumin.

FIG. 2 illustrates standard curves generated for human serum albumin using methods of the present disclosure.

FIG. 3 illustrates how the method of the present disclosure for human serum albumin correlates to the Beckman Coulter IMMAGE® assay for HAS.

FIG. 4 illustrates one embodiment of the present disclosure directed to vitamin D.

FIG. 5 illustrates standard curves generated for vitamin D using methods of the present disclosure.

FIG. 6A illustrates one embodiment of the present disclosure directed to C-reactive protein.

FIG. 6B illustrates one embodiment of the present disclosure directed to C-reactive protein.

FIG. 6C illustrates one embodiment of the present disclosure directed to C-reactive protein.

FIG. 7A illustrates a standard curve generated for C-reactive protein using methods of the present disclosure.

FIG. 7B illustrates the TR-FRET C-reactive protein assay reaches equilibrium after 1 minute.

FIG. 7C illustrates a standard curve generated for C-reactive protein using methods of the present disclosure.

FIG. 7D illustrates the correlation in calculated CRP when testing fingerstick whole blood tested on Procise vs. matched pair serum sample testing on the Orion QuikRead go CRP assay.

FIGS. 8A and 8B illustrate embodiments of the present disclosure directed to anti-transglutaminase autoantibody (ATA) immunoglobulin A (IgA) or G (IgG).

FIG. 9 illustrates a standard curve generated using methods of the present disclosure directed to ATA IgA.

FIG. 10A illustrates an embodiment of the present disclosure directed to anti-transglutaminase autoantibody (ATA) immunoglobulin A (IgA) or G (IgG).

FIG. 10B illustrates a standard curve generated using methods of the present disclosure directed to ATA IgA.

FIG. 11A illustrates the correlation between the present disclosure directed to anti-transglutaminase autoantibody (ATA) immunoglobulin (IgA) and the Inova ATA IgA assay.

FIG. 11B illustrates the sensitivity and specificity of the present disclosure for ATA IgA using a cut-off of 14 on 309 patient samples.

FIG. 12A illustrates the correlation between the present disclosure directed to anti-transglutaminase autoantibody (ATA) immunoglobulin (IgA) and the Phadia ATA IgA assay.

FIG. 12B illustrates the sensitivity and specificity of the present disclosure for ATA IgA using a cut-off of 9 on 355 patient samples.

FIG. 13 illustrates one embodiment of a donor fluorophore of the present disclosure.

FIG. 14 illustrates one embodiment of an acceptor fluorophore of the present disclosure.

FIG. 15 illustrates donor and acceptor wavelengths in one embodiment of the present disclosure.

### DETAILED DESCRIPTION

### I. Definitions

The terms “a,” “an,” or “the” as used herein not only includes aspects with one member, but also includes aspects with more than one member.

The term “about” as used herein to modify a numerical value indicates a defined range around that value. If “X” were the value, “about X” would indicate a value from 0.9X to 1.1X, and more preferably, a value from 0.95X to 1.05X. Any reference to “about X” specifically indicates at least the values X, 0.95X, 0.96X, 0.97X, 0.98X, 0.99X, 1.01X, 1.02X, 1.03X, 1.04X, and 1.05X. Thus, “about X” is intended to teach and provide written description support for a claim limitation of, e.g., “0.98X.”

When the modifier “about” is applied to describe the beginning of a numerical range, it applies to both ends of the range. Thus, “from about 500 to 850 nm” is equivalent to “from about 500 nm to about 850 nm.” When “about” is applied to describe the first value of a set of values, it applies to all values in that set. Thus, “about 580, 700, or 850 nm” is equivalent to “about 580 nm, about 700 nm, or about 850 nm.”

“Activated acyl” as used herein includes a —C(O)-LG group. “Leaving group” or “LG” is a group that is susceptible to displacement by a nucleophilic acyl substitution (i.e., a nucleophilic addition to the carbonyl of —C(O)-LG, followed by elimination of the leaving group). Representative leaving groups include halo, cyano, azido, carboxylic acid derivatives such as t-butylcarboxy, and carbonate derivatives such as i-BuOC(O)O—. An activated acyl group may also be an activated ester as defined herein or a carboxylic acid activated by a carbodiimide to form an anhydride (preferentially cyclic) or mixed anhydride —OC(O)R^{a} or —OC(NR^{a})NHR^{b} (preferably cyclic), wherein R^{a} and R^{b} are members independently selected from the group consisting of C₁-C₆ alkyl, C₁-C₆ perfluoroalkyl, C₁-C₆ alkoxy, cyclohexyl, 3-dimethylaminopropyl, or N-morpholinoethyl. Preferred activated acyl groups include activated esters.

“Activated ester” as used herein includes a derivative of a carboxyl group that is more susceptible to displacement by nucleophilic addition and elimination than an ethyl ester group (e.g., an NHS ester, a sulfo-NHS ester, a PAM ester, or a halophenyl ester). Representative carbonyl substituents of activated esters include succinimidyloxy (—OC₄H₄NO₂), sulfosuccinimidyloxy (—OC₄H₃NO₂SO₃H), -1-oxybenzotriazolyl (—OC₆H₄N₃); 4-sulfo-2,3,5,6-tetrafluorophenyl; or an aryloxy group that is optionally substituted one or more times by electron-withdrawing substituents such as nitro, fluoro, chloro, cyano, trifluoromethyl, or combinations thereof (e.g., pentafluorophenyloxy, or 2,3,5,6-tetrafluorophenyloxy). Preferred activated esters include succinimidyloxy, sulfosuccinimidyloxy, and 2,3,5,6-tetrafluorophenyloxy esters.

“FRET partners” refers to a pair of fluorophores consisting of a donor fluorescent compound such as cryptate and an acceptor compound such as Alexa 647, when they are in proximity to one another and when they are excited at the excitation wavelength of the donor fluorescent compound, these compounds emit a FRET signal. It is known that, in order for two fluorescent compounds to be FRET partners, the emission spectrum of the donor fluorescent compound must partially overlap the excitation spectrum of the acceptor compound. The preferred FRET-partner pairs are those for which the value R0 (Förster distance, distance at which energy transfer is 50% efficient) is greater than or equal to 30 Å.

“FRET signal” refers to any measurable signal representative of FRET between a donor fluorescent compound and an acceptor compound. A FRET signal can therefore be a variation in the intensity or in the lifetime of luminescence of the donor fluorescent compound or of the acceptor compound when the latter is fluorescent.

“Human serum albumin” refers to a protein that is in the human blood plasma and synthesized in the liver as a proalbumin precursor protein. The N-terminal peptide of the proalbumin precursor protein is removed to generate proalbumin, which is released from the rough endoplasmic reticulum into the Golgi vesicles where it is cleaved again to produce the matured serum albumin. Human serum albumin performs a number of functions in the blood, such as hormone and fatty acid transports and pH buffer. Human serum albumin has a molecular weight of approximately 66.5 kDa and a serum half-life of approximately 20 days. Human serum albumin, UniProt ID No. P02768, is SEQ ID NO: 1.

“Vitamin D” refers to a group of fat-soluble secosteroids, the most important of which are vitamin D2 (also known as ergocalciferol) and vitamin D3 (also known as cholecalciferol). Other compounds in the group are vitamin D1 (a mixture of ergocalciferol with lumisterol), vitamin D4 (22-dihydroergocalciferol), and vitamin D5 (sitocalciferol).

The chemical structures of vitamin D2 and vitamin D3 are shown below:

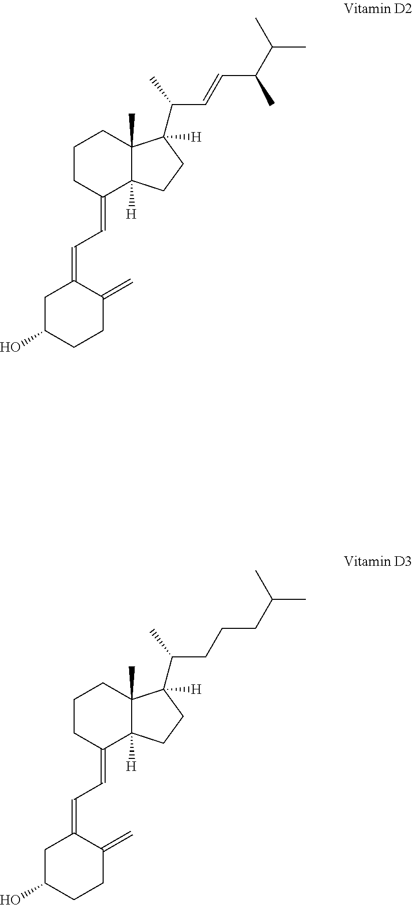 [SMILES: C=C1CC[C@H](O)C/C1=C/C=C1\CCC[C@]2(C)[C@@H]([C@H](C)/C=C/[C@H](C)C(C)C)CC[C@@H]12 ; C=C1CC[C@H](O)C/C1=C/C=C1\CCC[C@]2(C)[C@@H]([C@H](C)CCCC(C)C)CC[C@@H]12]

“C-reactive protein” or CRP refers to a pentameric protein found in the blood plasma, whose circulating concentrations rise in response to inflammation. The protein is synthesized by the liver in response to factors released by macrophages and fat cells (adipocytes). The C-reactive protein gene is located on chromosome 1 (1q23.2). Each monomer of its pentameric structure has 224 amino acids, and a molecular mass of 25,106 Da. In serum, it assembles into stable pentameric structure with a discoid shape. Human C-reactive protein, UniProt ID No. P02741, is SEQ ID NO: 2.

“Gluten-induced disease” as used herein relates to any gluten-induced disease entity or disorder such as Celiac disease, which is associated to autoantibodies against tissue transglutaminase tTG. Gluten-induced diseases often cause enteropathy and the most well-known gluten-induced diseases are celiac disease and dermatitis herpetiformis. However, there are also gluten-induced diseases without symptoms of enteropathy. Therefore a better indicator of a gluten-induced disease is the occurrence of tTG autoantibodies.

“Celiac disease (CD)” refers to a disease of the intestinal mucosa and is usually manifested in infants and children. CD is associated with an inflammation of the mucosa, which causes malabsorption. Individuals with celiac disease do not tolerate a protein called gluten, which is present in wheat, rye, barley and possibly oats. When exposed to gluten, the immune system of an individual with CD responds by attacking the lining of the small intestine. The only treatment of CD is a gluten-free diet, which usually results in morphological and clinical improvement.

“Transglutaminases (EC 2.3.2.13)” refers to a diverse family of Ca²⁺ dependent enzymes that are highly ubiquitous and highly conserved across species. Transglutaminases catalyze the covalent cross-linking of specific proteins through the formation of isopeptide bonds between α-carboxyl groups of glutamine residues in one polypeptide and ε-NH₂ groups of lysine residues in another. The resulting polymer network is stable and resistant or proteolysis, increasing the resistance of tissue to chemical, enzymatic and mechanical disruption. Of all the transglutaminases, tissue transglutaminases (tTG) is the most widely distributed. tTG provides the focus of the autoimmune response in CD.

“Anti-transglutaminase antibody” or “ATA” are autoantibodies against the transglutaminase protein. ATA IgGs and ATA IgAs are ATAs classified according to immunoglobulin reactivity subclass (IgA, IgG).

### II. Embodiments

Human Serum Albumin

Human serum albumin is one of the most abundant proteins in human blood plasma. Human serum albumin is synthesized in the liver as a proalbumin precursor protein that has an N-terminal peptide that is removed before the nascent protein is released from the rough endoplasmic reticulum as a proalbumin. The proalbumin is then cleaved in the Golgi vesicles to produce the matured serum albumin. Human serum albumin has a molecular weight of approximately 66.5 kDa and a serum half-life of approximately 20 days. Human serum albumin serves many functions in the blood, such as hormone and fatty acid transports and pH buffer. The presence and concentration level of human serum albumin is typically measured by an enzyme-linked immunosorbent assay (ELISA).

Human serum albumin solid-phase sandwich ELISA is designed to measure the presence or amount of the analyte bound between an antibody pair. In the sandwich ELISA, a sample is added to an immobilized capture antibody. After a second (detector) antibody is added, a substrate solution is used that reacts with an enzyme-antibody-target complex to produce a measurable signal. The intensity of this signal is proportional to the concentration of target present in the test sample.

The present disclosure provides a homogenous solution phase time-resolved FRET assay (TR-FRET) to detect human serum albumin presence or level in a biological sample such as whole blood. In conjunction with other markers levels, human serum albumin can be used as an aid in determination of fibrosis in liver diseases such as NASH, Hepatitis C and Hepatitis B. Förster resonance energy transfer or fluorescence resonance energy transfer (FRET) is a process in which a donor molecule in an excited state transfers its excitation energy through dipole-dipole coupling to an acceptor fluorophore, when the two molecules are brought into close proximity, typically less than 10 nm such as, <9 nm, <8 nm, <7 nm, <6 nm, <5 nm, <4 nm, <3 nm, <2 nm, or less than <1 nm. Upon excitation at a characteristic wavelength, the energy absorbed by the donor is transferred to the acceptor, which in turn emits the energy. The level of light emitted from the acceptor fluorophore is proportional to the degree of donor acceptor complex formation.

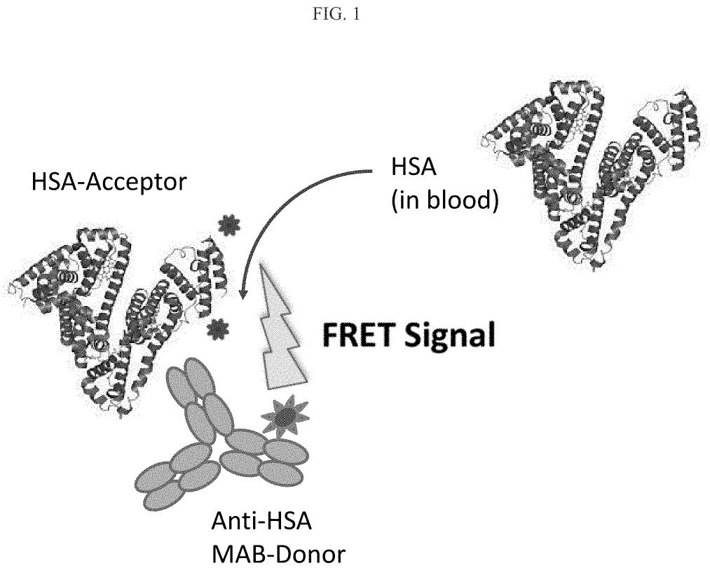

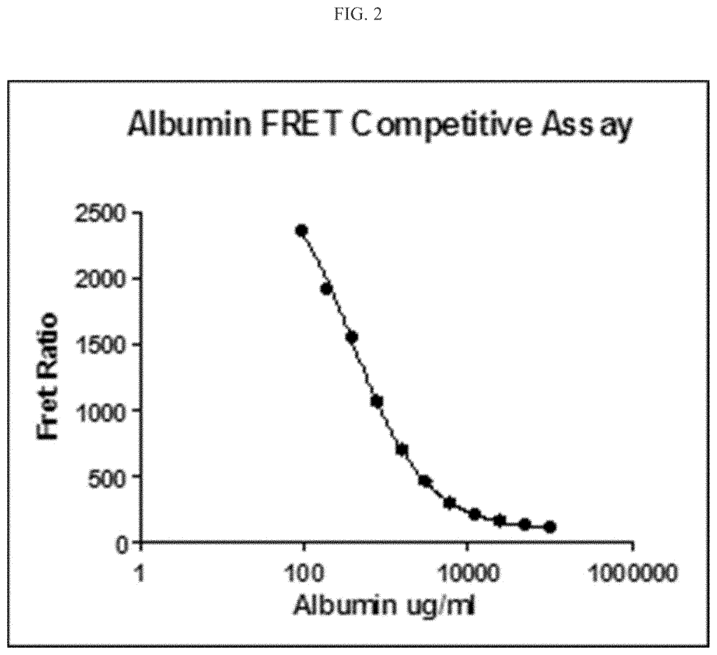

Biological materials are typically prone to autofluorescence, which can be minimized by utilizing time-resolved fluorometry (TRF). TRF takes advantage of unique rare earth elements such as lanthanides, (e.g., europium and terbium), which have exceptionally long fluorescence emission half-lives. Time-resolved FRET (TR-FRET) unites the properties of TRF and FRET, which is especially advantageous when analyzing biological samples. If an anti-human serum albumin antibody is labeled with a donor fluorophore and an isolated human serum albumin protein is labeled with an acceptor fluorophore, TR-FRET can occur in the presence of these two molecules which can form a complex via the binding of the anti-human serum albumin antibody to the isolated human serum albumin protein. Once this complex is in contact with a sample (e.g., a whole blood sample), the human serum albumin in the sample, if present, would compete with the isolated human serum albumin protein labeled with the acceptor fluorophore for binding with the anti-human serum albumin labeled with the donor fluorophore. Thus, if human serum albumin is present in the sample (e.g., a whole blood sample), it would disrupt the FRET signal initially emitted by the complex, leading to a decrease in the FRET signal (FIGS. 1 and 2). Therefore, in the presence of a low amount of human serum albumin in the sample (e.g., a whole blood sample), the FRET signal is near its maximum, as the isolated human serum albumin labeled with an acceptor fluorophore (or a donor fluorophore) binds unimpeded to the anti-human serum albumin antibody labeled with a donor fluorophore (or an acceptor fluorophore). In the presence of a high amount of human serum albumin, the FRET signal is low, since the human serum albumin in the sample (e.g., a whole blood sample) blocks or competes with the binding of the isolated human serum albumin to the anti-human serum albumin antibody. In some embodiments, the measurement of the presence or level of human serum albumin in a sample (e.g., a whole blood sample) by TR-FRET may be used as an aid to determine subject's malnutrition.

The use of the FRET phenomenon for studying biological processes implies that each member of the pair of FRET partners will be conjugated to compounds that will interact with one another, and thus bring the FRET partners into close proximity with one another. Upon exposure to light, the FRET partners will generate a FRET signal. In the methods according to the disclosure, the energy donor is conjugated to an anti-human serum albumin antibody and the energy acceptor is conjugated to an isolated human serum albumin protein. Alternatively, the energy acceptor is conjugated to an anti-human serum albumin antibody and the energy donor is conjugated to an isolated human serum albumin protein. The energy transfer between the two FRET partners depends upon the binding of the anti-human serum albumin to the isolated human serum albumin protein. Förster or fluorescence resonance energy transfer (FRET), is a physical phenomenon in which a donor fluorophore in its excited state non-radiatively transfers its excitation energy to a neighboring acceptor fluorophore, thereby causing the acceptor to emit its characteristic fluorescence.

As such, in one aspect, the present disclosure provides an assay method for detecting the presence or amount of human serum albumin in a sample, the method comprising:

contacting the sample with a complex comprising an anti-human serum albumin antibody labeled with a donor fluorophore and an isolated human serum albumin labeled with an acceptor fluorophore, wherein the complex emits a fluorescence emission signal associated with fluorescence resonance energy transfer (FRET) when the donor fluorophore is excited using a light source;

incubating the sample with the complex for a time sufficient for human serum albumin in the sample to compete for binding to the anti-human serum albumin antibody

labeled with the donor fluorophore; and exciting the sample using a light source to detect the fluorescence emission signal associated with FRET,

wherein an absence of the fluorescence emission signal or a decrease in the fluorescence emission signal relative to the fluorescence emission signal initially emitted by the complex indicates the presence or amount of human serum albumin in the sample.

In another aspect, the anti-human serum albumin can be labeled with an acceptor fluorophore and an isolated human serum albumin can be labeled with a donor fluorophore in an assay method.

In both aspects of the disclosure, it is the absence, disappearance, or decrease in the fluorescence emission signal relative to the fluorescence emission signal initially emitted by the complex that indicates the presence or amount of human serum albumin in the sample. The donor fluorophore in its excited state can transfer its excitation energy to the acceptor fluorophore to cause the acceptor fluorophore to emit its characteristic fluorescence. However, if human serum albumin is present in the sample and competes with the isolated human serum albumin for binding to the anti-human serum albumin antibody, then the fluorescence energy transfer between the donor and acceptor fluorophores would be disrupted, leading to a loss or decrease of the fluorescence emission signal.

In certain aspects, the FRET assay is a time-resolved FRET assay. The fluorescence emission signal or measured FRET signal is directly correlated with the biological phenomenon studied. In fact, the level of energy transfer between the donor fluorescent compound and the acceptor fluorescent compound is proportional to the reciprocal of the distance between these compounds to the 6th power. For the donor/acceptor pairs commonly used by those skilled in the art, the distance R0 (corresponding to a transfer efficiency of 50%) is in the order of 1, 5, 10, 20 or 30 nanometers. Specifically in the methods described herein, the decrease in FRET signal as the complex, which is formed by the binding of the anti-human serum albumin labeled with a donor fluorophore (or an acceptor fluorophore) to the isolated human serum albumin protein labeled with an acceptor fluorophore (or a donor fluorophore), comes in contact with the sample (e.g., a whole blood sample) relative to the initial FRET signal emitted by the complex prior to the complex is in contact with the sample is correlated with the presence of human serum albumin in the sample (see, e.g., FIGS. 1 and 2).

In certain aspects, the sample is a biological sample. Suitable biological samples include, but are not limited to, whole blood, urine, a fecal specimen, plasma or serum. In a preferred aspect, the biological sample is whole blood.

In certain aspects, the FRET energy donor compound is a cryptate, such as a lanthanide cryptate.

In certain aspects, the cryptate has an absorption wavelength between about 300 nm to about 400 nm such as about 325 nm to about 375 nm. In certain aspects, as shown in FIG. 15, cyptate dyes (Lumi4-Tb in FIG. 15) have four fluorescence emission peaks at about 490 nm, about 548 nm, about 587 nm, and 621 nm. Thus, as a donor, the cryptate is compatible with fluorescein-like (green zone) and Cy5 or DY-647-like (red zone) acceptor (e.g., green acceptor, NIR acceptor, or orange acceptor in FIG. 15) to perform TR-FRET experiments.

In certain aspects, the introduction of a time delay between a flash excitation and the measurement of the fluorescence at the acceptor emission wavelength allows to discriminate long lived from short-lived fluorescence and to increase signal-to-noise ratio.

Vitamin D

Vitamin D is a group of fat-soluble secosteroid, the most important of which are vitamin D2 (also known as ergocalciferol) and vitamin D3 (also known as cholecalciferol). Cholecalciferol and ergocalciferol can be ingested from the diet and from supplements. Only a few foods contain vitamin D, such as fish, eggs, and fortified dairy products. A major natural source of the vitamin is the synthesis of cholecalciferol in the skin from cholesterol through a chemical reaction that is dependent on sun exposure (specifically UVB radiation). Vitamin D performs a number of functions, such as increasing intestinal absorption of calcium, magnesium, and phosphate, and promoting bone growth. The presence and concentration level of vitamin D is typically measured by an enzyme-linked immunosorbent assay (ELISA).

Vitamin D solid-phase sandwich ELISA is designed to measure the presence or amount of the analyte bound between an antibody pair. In the sandwich ELISA, a sample is added to an immobilized capture antibody. After a second (detector) antibody is added, a substrate solution is used that reacts with an enzyme-antibody-target complex to produce a measurable signal. The intensity of this signal is proportional to the concentration of target present in the test sample.

The present disclosure provides a homogenous solution phase time-resolved FRET assay (TR-FRET) to detect vitamin D presence or level in a biological sample such as whole blood. In conjunction with other markers levels, vitamin D can be used as an aid in determination of fibrosis in liver diseases such as NASH, Hepatitis C and Hepatitis B. Förster resonance energy transfer or fluorescence resonance energy transfer (FRET) is a process in which a donor molecule in an excited state transfers its excitation energy through dipole-dipole coupling to an acceptor fluorophore, when the two molecules are brought into close proximity, typically less than 10 nm such as, <9 nm, <8 nm, <7 nm, <6 nm, <5 nm, <4 nm, <3 nm, <2 nm, or less than <1 nm. Upon excitation at a characteristic wavelength, the energy absorbed by the donor is transferred to the acceptor, which in turn emits the energy. The level of light emitted from the acceptor fluorophore is proportional to the degree of donor acceptor complex formation.

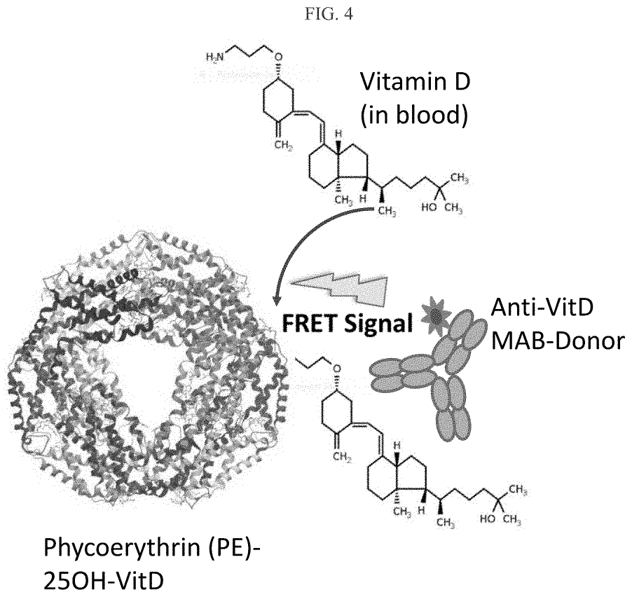

Biological materials are typically prone to autofluorescence, which can be minimized by utilizing time-resolved fluorometry (TRF). TRF takes advantage of unique rare earth elements such as lanthanides, (e.g., europium and terbium), which have exceptionally long fluorescence emission half-lives. Time-resolved FRET (TR-FRET) unites the properties of TRF and FRET, which is especially advantageous when analyzing biological samples. If a vitamin D-binding agent is labeled with a donor fluorophore and an isolated vitamin D is labeled with an acceptor fluorophore, TR-FRET can occur in the presence of these two molecules which can form a complex via the binding of the vitamin D-binding agent to the isolated vitamin D. Once this complex is in contact with a sample (e.g., a whole blood sample), the vitamin D in the sample, if present, would compete with the isolated vitamin D labeled with the acceptor fluorophore for binding with the vitamin D-binding agent labeled with the donor fluorophore. Thus, if vitamin D is present in the sample (e.g., a whole blood sample), it would disrupt the FRET signal initially emitted by the complex, leading to a decrease in the FRET signal (FIGS. 4 and 5). Therefore, in the presence of a low amount of vitamin D in the sample (e.g., a whole blood sample), the FRET signal is near its maximum, as the isolated vitamin D labeled with an acceptor fluorophore (or a donor fluorophore) binds unimpeded to the vitamin D-binding agent labeled with a donor fluorophore (or an acceptor fluorophore). In the presence of a high amount of vitamin D, the FRET signal is low, since the vitamin D in the sample (e.g., a whole blood sample) blocks or competes with the binding of the isolated vitamin D to the vitamin D-binding agent.

The use of the FRET phenomenon for studying biological processes implies that each member of the pair of FRET partners will be conjugated to compounds that will interact with one another, and thus bring the FRET partners into close proximity with one another. Upon exposure to light, the FRET partners will generate a FRET signal. In the methods according to the disclosure, the energy donor is conjugated to a vitamin D-binding agent and the energy acceptor is conjugated to an isolated vitamin D. Alternatively, the energy acceptor is conjugated to a vitamin D-binding agent and the energy donor is conjugated to an isolated vitamin D. The energy transfer between the two FRET partners depends upon the binding of the vitamin D-binding agent to the isolated vitamin D. Förster or fluorescence resonance energy transfer (FRET), is a physical phenomenon in which a donor fluorophore in its excited state non-radiatively transfers its excitation energy to a neighboring acceptor fluorophore, thereby causing the acceptor to emit its characteristic fluorescence.

As such, in one aspect, the present disclosure provides an assay method for detecting the presence or amount of vitamin D in a sample, the method comprising:

contacting the sample with a complex comprising a vitamin D-binding agent labeled with a donor fluorophore and an isolated vitamin D labeled with an acceptor fluorophore, wherein the complex emits a fluorescence emission signal associated with fluorescence resonance energy transfer (FRET) when the donor fluorophore is excited using a light source;

incubating the sample with the complex for a time sufficient for vitamin D in the sample to compete for binding to the vitamin D-binding agent labeled with the donor fluorophore; and

exciting the sample using a light source to detect the fluorescence emission signal associated with FRET,

wherein an absence of the fluorescence emission signal or a decrease in the fluorescence emission signal relative to the fluorescence emission signal initially emitted by the complex indicates the presence or amount of vitamin D in the sample.

In another aspect, the vitamin D-binding agent can be labeled with an acceptor fluorophore and an isolated vitamin D can be labeled with a donor fluorophore in an assay method.

In both aspects of the disclosure, it is the absence, disappearance, or decrease in the fluorescence emission signal relative to the fluorescence emission signal initially emitted by the complex that indicates the presence or amount of vitamin D in the sample. The donor fluorophore in its excited state can transfer its excitation energy to the acceptor fluorophore to cause the acceptor fluorophore to emit its characteristic fluorescence. However, if vitamin D is present in the sample and competes with the isolated vitamin D for binding to the vitamin D-binding agent, then the fluorescence energy transfer between the donor and acceptor fluorophores would be disrupted, leading to a loss or decrease of the fluorescence emission signal.

In certain aspects, the FRET assay is a time-resolved FRET assay. The fluorescence emission signal or measured FRET signal is directly correlated with the biological phenomenon studied. In fact, the level of energy transfer between the donor fluorescent compound and the acceptor fluorescent compound is proportional to the reciprocal of the distance between these compounds to the 6th power. For the donor/acceptor pairs commonly used by those skilled in the art, the distance R0 (corresponding to a transfer efficiency of 50%) is in the order of 1, 5, 10, 20 or 30 nanometers. Specifically in the methods described herein, the decrease in FRET signal as the complex, which is formed by the binding of the vitamin D-binding agent labeled with a donor fluorophore (or an acceptor fluorophore) to the isolated vitamin D labeled with an acceptor fluorophore (or a donor fluorophore), comes in contact with the sample (e.g., a whole blood sample) relative to the initial FRET signal emitted by the complex prior to the complex is in contact with the sample is correlated with the presence of vitamin D in the sample (see, e.g., FIGS. 4 and 5).

In certain aspects, the sample is a biological sample. Suitable biological samples include, but are not limited to, whole blood, urine, a fecal specimen, plasma or serum. In a preferred aspect, the biological sample is whole blood.

In certain aspects, the FRET energy donor compound is a cryptate, such as a lanthanide cryptate.

In certain aspects, the cryptate has an absorption wavelength between about 300 nm to about 400 nm such as about 325 nm to about 375 nm. In certain aspects, as shown in FIG. 15, cyptate dyes (Lumi4-Tb in FIG. 15) have four fluorescence emission peaks at about 490 nm, about 548 nm, about 587 nm, and 621 nm. Thus, as a donor, the cryptate is compatible with fluorescein-like (green zone) and Cy5 or DY-647-like (red zone) acceptor (e.g., green acceptor, NIR acceptor, or orange acceptor in FIG. 15) to perform TR-FRET experiments.

In certain aspects, the introduction of a time delay between a flash excitation and the measurement of the fluorescence at the acceptor emission wavelength allows to discriminate long lived from short-lived fluorescence and to increase signal-to-noise ratio.

C-Reactive Protein

C-reactive protein is a pentameric protein found in the blood plasma, whose circulating concentrations rise in response to inflammation. The protein is synthesized by the liver in response to factors released by macrophages and fat cells (adipocytes). C-reactive protein binds to lysophosphatidylcholine expressed on the surface of dead or dying cells (and some types of bacteria) in order to activate the complement system via C1q. The C-reactive protein gene is located on chromosome 1 (1q23.2). Each monomer of its pentameric structure has 224 amino acids, and a molecular mass of 25,106 Da. In serum, it assembles into stable pentameric structure with a discoid shape. The presence and concentration level of C-reactive protein is typically measured by an enzyme-linked immunosorbent assay (ELISA).

C-reactive protein solid-phase sandwich ELISA is designed to measure the presence or amount of the analyte bound between an antibody pair. In the sandwich ELISA, a sample is added to an immobilized capture antibody. After a second (detector) antibody is added, a substrate solution is used that reacts with an enzyme-antibody-target complex to produce a measurable signal. The intensity of this signal is proportional to the concentration of target present in the test sample.

The present disclosure provides a homogenous solution phase time-resolved FRET assay (TR-FRET) to detect C-reactive protein presence or level in a biological sample such as whole blood. In conjunction with other markers levels, C-reactive protein can be used as an aid in determination of fibrosis in liver diseases such as NASH, Hepatitis C and Hepatitis B. Förster resonance energy transfer or fluorescence resonance energy transfer (FRET) is a process in which a donor molecule in an excited state transfers its excitation energy through dipole-dipole coupling to an acceptor fluorophore, when the two molecules are brought into close proximity, typically less than 10 nm such as, <9 nm, <8 nm, <7 nm, <6 nm, <5 nm, <4 nm, <3 nm, <2 nm, or less than <1 nm. Upon excitation at a characteristic wavelength, the energy absorbed by the donor is transferred to the acceptor, which in turn emits the energy. The level of light emitted from the acceptor fluorophore is proportional to the degree of donor acceptor complex formation.

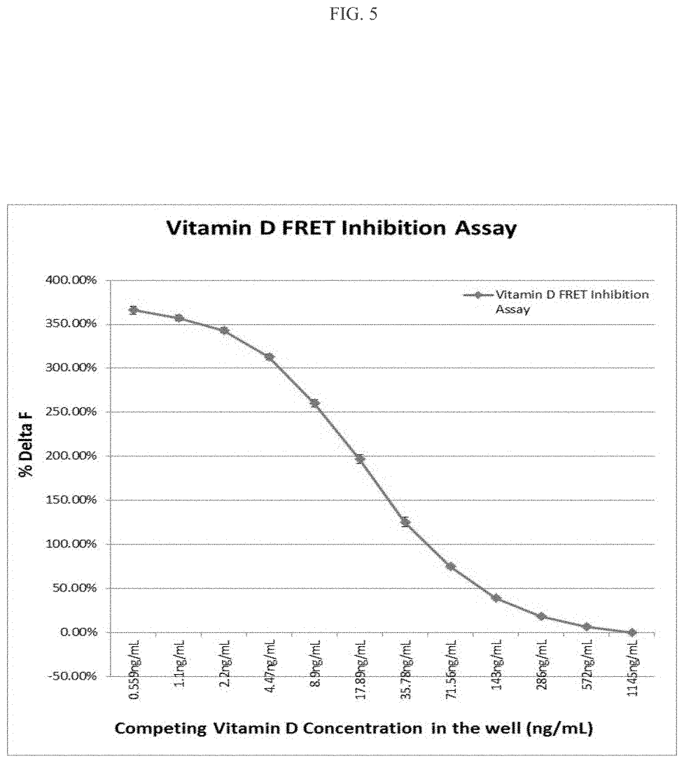

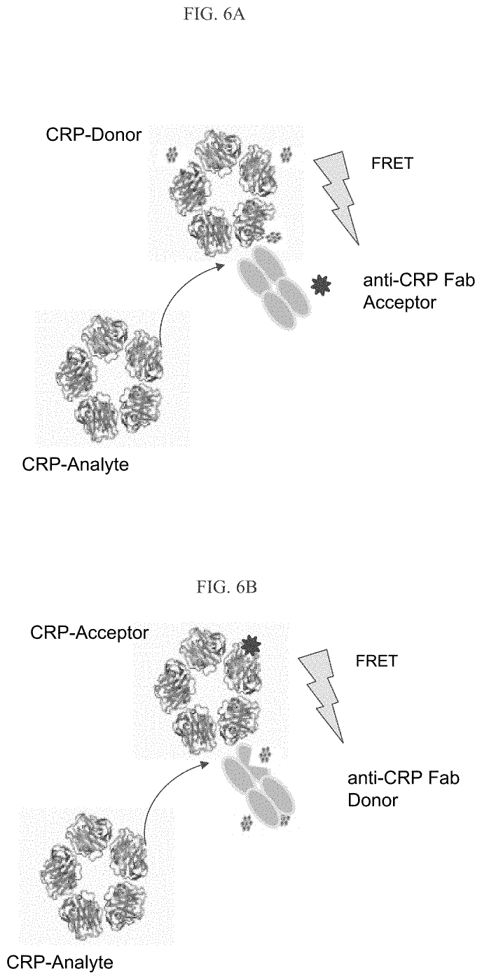

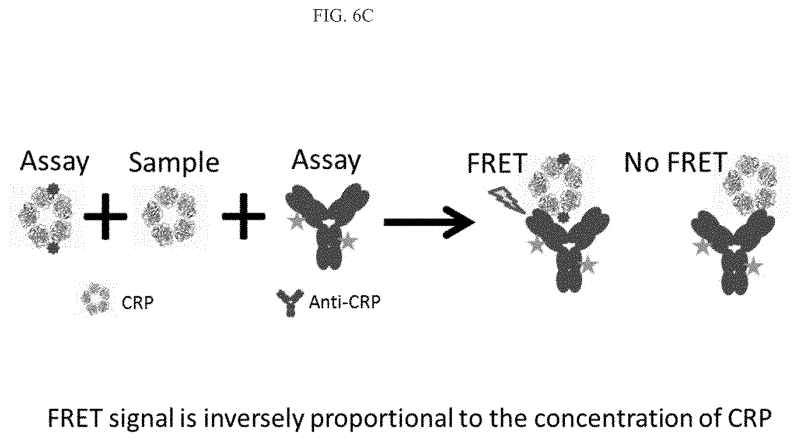

Biological materials are typically prone to autofluorescence, which can be minimized by utilizing time-resolved fluorometry (TRF). TRF takes advantage of unique rare earth elements such as lanthanides, (e.g., europium and terbium), which have exceptionally long fluorescence emission half-lives. Time-resolved FRET (TR-FRET) unites the properties of TRF and FRET, which is especially advantageous when analyzing biological samples. If an anti-C-reactive protein antibody is labeled with a donor fluorophore and an isolated C-reactive protein is labeled with an acceptor fluorophore, TR-FRET can occur in the presence of these two molecules which can form a complex via the binding of the anti-C-reactive protein antibody to the isolated C-reactive protein. Once this complex is in contact with a sample (e.g., a whole blood sample), the C-reactive protein in the sample, if present, would compete with the isolated C-reactive protein labeled with the acceptor fluorophore for binding with the anti-C-reactive protein labeled with the donor fluorophore. Thus, if C-reactive protein is present in the sample (e.g., a whole blood sample), it would disrupt the FRET signal initially emitted by the complex, leading to a decrease in the FRET signal (FIG. 6A and FIG. 6B). Therefore, in the presence of a low amount of C-reactive protein in the sample (e.g., a whole blood sample), the FRET signal is near its maximum, as the isolated C-reactive protein labeled with an acceptor fluorophore (or a donor fluorophore) binds unimpeded to the anti-C-reactive protein antibody labeled with a donor fluorophore (or an acceptor fluorophore). In the presence of a high amount of C-reactive protein, the FRET signal is low, since the C-reactive protein in the sample (e.g., a whole blood sample) blocks or competes with the binding of the isolated C-reactive protein to the anti-C-reactive protein antibody. In some embodiments, the measurement of the presence or level of C-reactive protein in a sample (e.g., a whole blood sample) by TR-FRET may be used as an aid to determine subject's malnutrition.

The use of the FRET phenomenon for studying biological processes implies that each member of the pair of FRET partners will be conjugated to compounds that will interact with one another, and thus bring the FRET partners into close proximity with one another. Upon exposure to light, the FRET partners will generate a FRET signal. In the methods according to the disclosure, the energy donor is conjugated to an anti-C-reactive protein antibody and the energy acceptor is conjugated to an isolated C-reactive protein. Alternatively, the energy acceptor is conjugated to an anti-C-reactive protein antibody and the energy donor is conjugated to an isolated C-reactive protein. The energy transfer between the two FRET partners depends upon the binding of the anti-C-reactive protein to the isolated C-reactive protein. Förster or fluorescence resonance energy transfer (FRET), is a physical phenomenon in which a donor fluorophore in its excited state non-radiatively transfers its excitation energy to a neighboring acceptor fluorophore, thereby causing the acceptor to emit its characteristic fluorescence.

As such, in one aspect, the present disclosure provides an assay method for detecting the presence or amount of C-reactive protein in a sample, the method comprising:

contacting the sample with a complex comprising an anti-C-reactive protein antibody labeled with a donor fluorophore and an isolated C-reactive protein labeled with an acceptor fluorophore, wherein the complex emits a fluorescence emission signal associated with fluorescence resonance energy transfer (FRET) when the donor fluorophore is excited using a light source;

incubating the sample with the complex for a time sufficient for C-reactive protein in the sample to compete for binding to the anti-C-reactive protein antibody labeled with the donor fluorophore; and

exciting the sample using a light source to detect the fluorescence emission signal associated with FRET,

wherein an absence of the fluorescence emission signal or a decrease in the fluorescence emission signal relative to the fluorescence emission signal initially emitted by the complex indicates the presence or amount of C-reactive protein in the sample.

In another aspect, the anti-C-reactive protein can be labeled with an acceptor fluorophore and an isolated C-reactive protein can be labeled with a donor fluorophore in an assay method.

In both aspects of the disclosure, it is the absence, disappearance, or decrease in the fluorescence emission signal relative to the fluorescence emission signal initially emitted by the complex that indicates the presence or amount of C-reactive protein in the sample. The donor fluorophore in its excited state can transfer its excitation energy to the acceptor fluorophore to cause the acceptor fluorophore to emit its characteristic fluorescence. However, if C-reactive protein is present in the sample and competes with the isolated C-reactive protein for binding to the anti-C-reactive protein antibody, then the fluorescence energy transfer between the donor and acceptor fluorophores would be disrupted, leading to a loss or decrease of the fluorescence emission signal.

In certain aspects, the FRET assay is a time-resolved FRET assay. The fluorescence emission signal or measured FRET signal is directly correlated with the biological phenomenon studied. In fact, the level of energy transfer between the donor fluorescent compound and the acceptor fluorescent compound is proportional to the reciprocal of the distance between these compounds to the 6th power. For the donor/acceptor pairs commonly used by those skilled in the art, the distance R0 (corresponding to a transfer efficiency of 50%) is in the order of 1, 5, 10, 20 or 30 nanometers. Specifically in the methods described herein, the decrease in FRET signal as the complex, which is formed by the binding of the anti-C-reactive protein labeled with a donor fluorophore (or an acceptor fluorophore) to the isolated C-reactive protein labeled with an acceptor fluorophore (or a donor fluorophore), comes in contact with the sample (e.g., a whole blood sample) relative to the initial FRET signal emitted by the complex prior to the complex is in contact with the sample is correlated with the presence of C-reactive protein in the sample (see, e.g., FIGS. 6A-6C).

In certain aspects, the sample is a biological sample. Suitable biological samples include, but are not limited to, whole blood, urine, a fecal specimen, plasma or serum. In a preferred aspect, the biological sample is whole blood.

In certain aspects, the FRET energy donor compound is a cryptate, such as a lanthanide cryptate.

In certain aspects, the cryptate has an absorption wavelength between about 300 nm to about 400 nm such as about 325 nm to about 375 nm. In certain aspects, as shown in FIG. 15, cyptate dyes (Lumi4-Tb in FIG. 15) have four fluorescence emission peaks at about 490 nm, about 548 nm, about 587 nm, and 621 nm. Thus, as a donor, the cryptate is compatible with fluorescein-like (green zone) and Cy5 or DY-647-like (red zone) acceptor (e.g., green acceptor, NIR acceptor, or orange acceptor in FIG. 15) to perform TR-FRET experiments.

In certain aspects, the introduction of a time delay between a flash excitation and the measurement of the fluorescence at the acceptor emission wavelength allows to discriminate long lived from short-lived fluorescence and to increase signal-to-noise ratio.

Anti-Transglutaminase Antibodies

Anti-transglutaminase antibodies (ATAs) are autoantibodies against the transglutaminase protein. Antibodies serve an important role in the immune system by detecting cells and substances that the rest of the immune system then eliminates. Antibodies against the body's own products are called autoantibodies. Autoantibodies can sometimes errantly be directed against healthy portions of the organism, causing autoimmune diseases. ATA can be classified according to two different schemes: transglutaminase isoform and immunoglobulin reactivity subclass (IgA, IgG) toward transglutaminases. Antibodies to tissue transglutaminase are often found in patients with several conditions, including celiac disease, juvenile diabetes, inflammatory bowel disease, and various forms of arthritis. In celiac disease, ATA are involved in the destruction of the villous extracellular matrix and target the destruction of intestinal villous epithelial cells by killer cells. Deposits of ATAs in the intestinal epithelium are often used to diagnose celiac disease. The presence and concentration level of ATAs is typically measured by an enzyme-linked immunosorbent assay (ELISA).

ATA solid-phase sandwich ELISA is designed to measure the presence or amount of the analyte bound between an antibody pair. In the sandwich ELISA, a sample is added to an immobilized capture antibody. After a second (detector) antibody is added, a substrate solution is used that reacts with an enzyme-antibody-target complex to produce a measurable signal. The intensity of this signal is proportional to the concentration of target present in the test sample.

The present disclosure provides a homogenous solution phase time-resolved FRET assay (TR-FRET) to detect ATA presence or level in a biological sample such as whole blood. In conjunction with other markers levels, ATA can be used as an aid in determination of fibrosis in liver diseases such as NASH, Hepatitis C and Hepatitis B. Förster resonance energy transfer or fluorescence resonance energy transfer (FRET) is a process in which a donor molecule in an excited state transfers its excitation energy through dipole-dipole coupling to an acceptor fluorophore, when the two molecules are brought into close proximity, typically less than 10 nm such as, <9 nm, <8 nm, <7 nm, <6 nm, <5 nm, <4 nm, <3 nm, <2 nm, or less than <1 nm. Upon excitation at a characteristic wavelength, the energy absorbed by the donor is transferred to the acceptor, which in turn emits the energy. The level of light emitted from the acceptor fluorophore is proportional to the degree of donor acceptor complex formation.

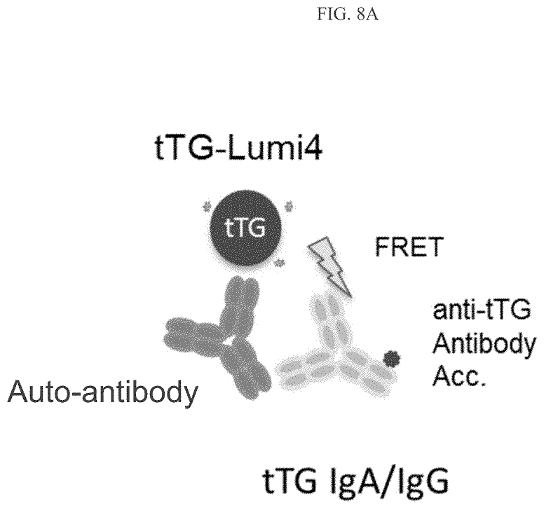

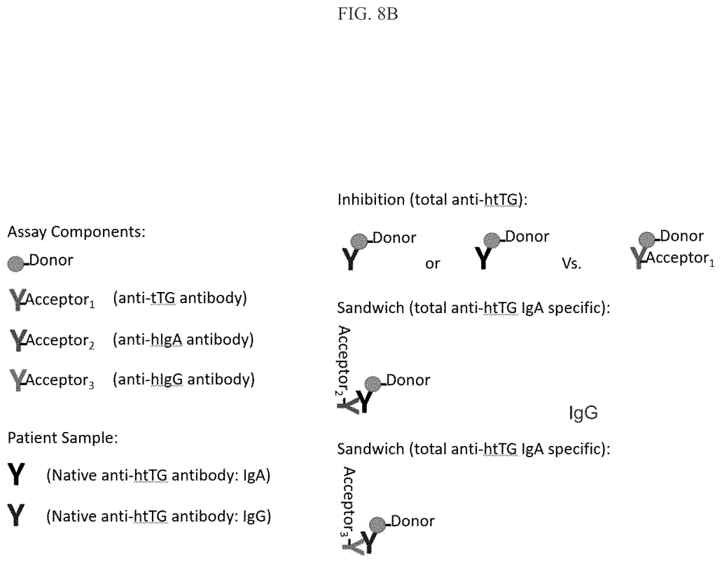

Biological materials are typically prone to autofluorescence, which can be minimized by utilizing time-resolved fluorometry (TRF). TRF takes advantage of unique rare earth elements such as lanthanides, (e.g., europium and terbium), which have exceptionally long fluorescence emission half-lives. Time-resolved FRET (TR-FRET) unites the properties of TRF and FRET, which is especially advantageous when analyzing biological samples. In one aspect, if an anti-tissue transglutaminase antibody is labeled with a donor fluorophore (or, alternatively, an acceptor fluorophore) and an isolated tissue transglutaminase protein is labeled with an acceptor fluorophore (or, alternatively, a donor fluorophore), TR-FRET can occur in the presence of these two molecules which can form a complex via the binding of the anti-tissue transglutaminase antibody to the isolated tissue transglutaminase protein. Once this complex is in contact with a sample (e.g., a whole blood sample), the endogenous anti-tissue transglutaminase antibody (autoantibody) in the sample, if present, would compete with the fluorophore labeled anti-tissue transglutaminase antibody for binding with the isolated tissue transglutaminase protein. Thus, if endogenous anti-tissue transglutaminase antibody (autoantibody) is present in the sample (e.g., a whole blood sample), it would disrupt the FRET signal initially emitted by the complex, leading to a decrease in the FRET signal (FIG. 8A). Therefore, in the presence of a low amount of endogenous anti-tissue transglutaminase antibody in the sample (e.g., a whole blood sample), the FRET signal is near its maximum, as the fluorophore-labeled anti-tissue transglutaminase antibody binds unimpeded to the isolated fluorophore-labeled tissue transglutaminase protein. In the presence of a high amount of endogenous anti-tissue transglutaminase antibody, the FRET signal is low, since the endogenous anti-tissue transglutaminase antibody in the sample (e.g., a whole blood sample) blocks or competes with the binding of the isolated tissue transglutaminase protein to the fluorophore-labeled anti-tissue transglutaminase antibody. In some embodiments, the measurement of the presence or level of endogenous anti-tissue transglutaminase antibody in a sample (e.g., a whole blood sample) by TR-FRET may be used as an aid to determine subject's disease condition.

The use of the FRET phenomenon for studying biological processes implies that each member of the pair of FRET partners will be conjugated to compounds that will interact with one another, and thus bring the FRET partners into close proximity with one another. Upon exposure to light, the FRET partners will generate a FRET signal. In the methods according to the disclosure, the energy donor (or acceptor) is conjugated to an anti-tissue transglutaminase antibody and the energy acceptor (or donor) is conjugated to an isolated tissue transglutaminase protein. The energy transfer between the two FRET partners depends upon the binding of the anti-tissue transglutaminase antibody to the isolated tissue transglutaminase protein. Förster or fluorescence resonance energy transfer (FRET), is a physical phenomenon in which a donor fluorophore in its excited state non-radiatively transfers its excitation energy to a neighboring acceptor fluorophore, thereby causing the acceptor to emit its characteristic fluorescence.

As such, in one aspect, the present disclosure provides an assay method for detecting the presence or amount of anti-transglutaminase autoantibody (ATA) immunoglobulin A (IgA) and/or ATA immunoglobulin G (IgG) in a sample, the method comprising:

contacting the sample with a complex comprising an anti-tissue transglutaminase antibody labeled with a donor fluorophore and an isolated tissue transglutaminase labeled with an acceptor fluorophore, wherein the anti-tissue transglutaminase antibody comprises a binding epitope to tissue transglutaminase, and wherein the complex emits a fluorescence emission signal associated with fluorescence resonance energy transfer (FRET) when the donor fluorophore is excited using a light source;

incubating the sample with the complex for a time sufficient for ATA IgA and/or ATA IgG in the sample to compete for binding to the isolated tissue transglutaminase labeled with the acceptor fluorophore; and

exciting the sample using a light source to detect a fluorescence emission signal associated with FRET,

wherein an absence of the fluorescence emission signal or a decrease in the fluorescence emission signal relative to the fluorescence emission signal initially emitted by the complex indicates the presence or amount of ATA IgA and/or ATA IgG in the sample.

In another aspect, the anti-tissue transglutaminase antibody can be labeled with an acceptor fluorophore and an isolated tissue transglutaminase protein can be labeled with a donor fluorophore in an assay method.

In both aspects of the disclosure, it is the absence, disappearance, or decrease in the fluorescence emission signal relative to the fluorescence emission signal initially emitted by the complex that indicates the presence or amount of endogenous anti-tissue transglutaminase antibody in the sample. The donor fluorophore in its excited state can transfer its excitation energy to the acceptor fluorophore to cause the acceptor fluorophore to emit its characteristic fluorescence. However, if endogenous anti-tissue transglutaminase antibody is present in the sample and competes with the fluorophore-labeled anti-tissue transglutaminase antibody for binding to the isolated tissue transglutaminase protein, then the fluorescence energy transfer between the donor and acceptor fluorophores would be disrupted, leading to a loss or decrease of the fluorescence emission signal.

In another aspect of the disclosure, the disclosure provides an assay method that can detect, as well as distinguish, the presence or amount of anti-transglutaminase autoantibody (ATA) immunoglobulin A (IgA) and ATA immunoglobulin G (IgG) in a sample (FIG. 8B), the method comprising:

contacting the sample with a complex comprising an anti-tissue transglutaminase antibody labeled with a donor fluorophore (or a first acceptor fluorophore) and an isolated tissue transglutaminase labeled with a first acceptor fluorophore (or a donor fluorophore), wherein the anti-tissue transglutaminase antibody comprises a binding epitope to tissue transglutaminase, and wherein the complex emits a fluorescence emission signal associated with fluorescence resonance energy transfer (FRET) when the donor fluorophore is excited using a light source;

incubating the sample with the complex for a time sufficient for ATA IgA and/or ATA IgG in the sample to compete for binding to the isolated tissue transglutaminase labeled with the first acceptor fluorophore (or a donor fluorophore);

further incubating the sample with an anti-IgA antibody labeled with a second acceptor fluorophore and an anti-IgG antibody labeled with a third acceptor fluorophore; and

exciting the sample using a light source to detect fluorescence emission signals associated with FRET,

wherein a detection of a fluorescence signal emitted by the second acceptor fluorophore indicates the presence or amount of ATA IgA and a detection of a fluorescence signal emitted by the third acceptor fluorophore indicates the presence or amount of ATA IgG in the sample.

In some embodiments of this aspect of the disclosure, prior to further incubating the sample with an anti-IgA antibody labeled with a second acceptor fluorophore and an anti-IgG antibody labeled with a third acceptor fluorophore, a total amount of ATA IgA and ATA IgG can be determined by exciting the sample using a light source to detect a fluorescence emission signal associated with FRET, wherein an absence of the fluorescence emission signal or a decrease in the fluorescence emission signal relative to the fluorescence emission signal initially emitted by the complex indicates the total amount of ATA IgA and ATA IgG in the sample.

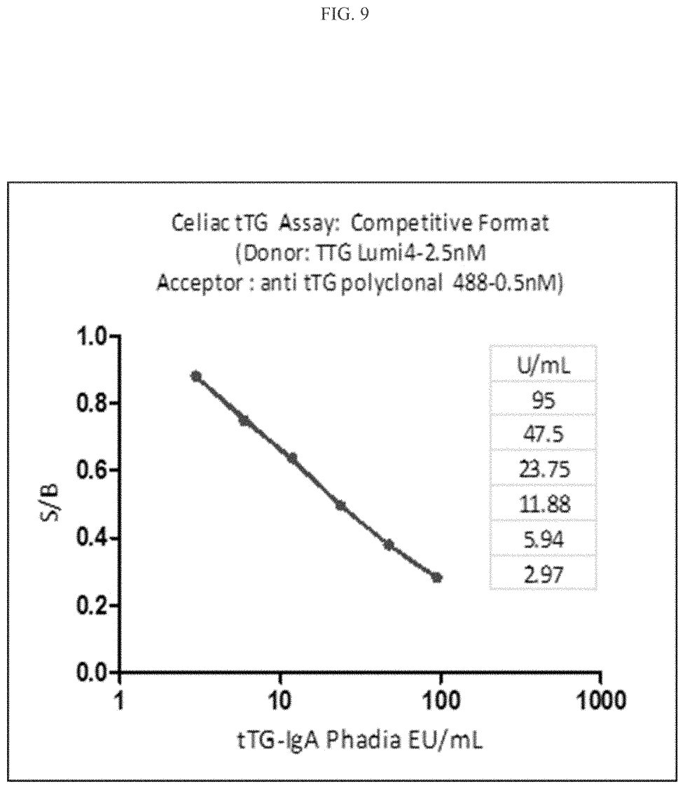

In certain aspects, the FRET assay is a time-resolved FRET assay. The fluorescence emission signal or measured FRET signal is directly correlated with the biological phenomenon studied. In fact, the level of energy transfer between the donor fluorescent compound and the acceptor fluorescent compound is proportional to the reciprocal of the distance between these compounds to the 6th power. For the donor/acceptor pairs commonly used by those skilled in the art, the distance R0 (corresponding to a transfer efficiency of 50%) is in the order of 1, 5, 10, 20 or 30 nanometers. Specifically in the methods described herein, the decrease in FRET signal as the complex, which is formed by the binding of the anti-tissue transglutaminase antibody labeled with a donor fluorophore (or an acceptor fluorophore) to the isolated tissue transglutaminase protein labeled with an acceptor fluorophore (or a donor fluorophore), comes in contact with the sample (e.g., a whole blood sample) relative to the initial FRET signal emitted by the complex prior to the complex is in contact with the sample is correlated with the presence of endogenous anti-tissue transglutaminase antibody in the sample (see, e.g., FIGS. 8A, 8B, and 9).

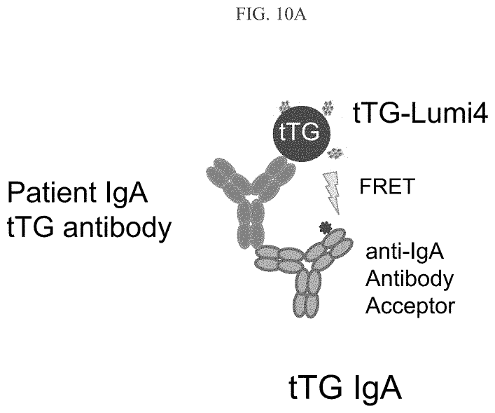

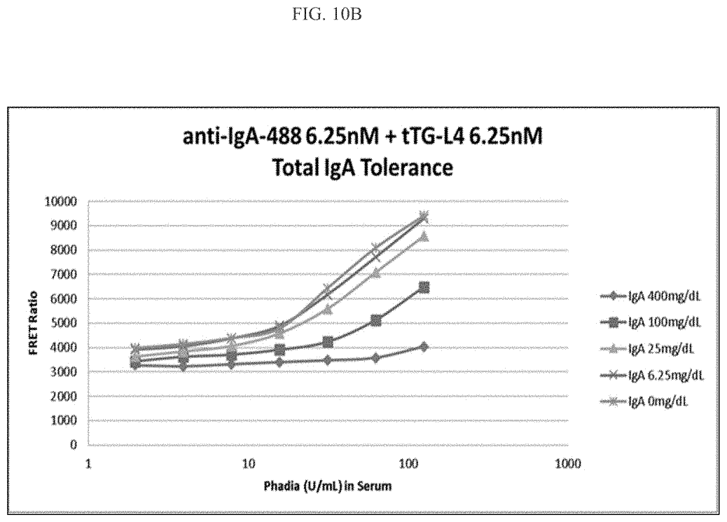

In another aspect, if an anti-IgA and/or anti-IgG antibody is labeled with a donor fluorophore (or, alternatively, an acceptor fluorophore) and an isolated tissue transglutaminase protein is labeled with an acceptor fluorophore (or, alternatively, a donor fluorophore), TR-FRET can occur in the presence of anti-tissue transglutaminase antibody IgA and/or IgG (ATA IgA and/or ATA IgG), which can bring the anti-IgA and/or anti-IgG antibody and the isolated tissue transglutaminase protein together to form a complex via the binding between the ATA IgA and/or ATA IgG and the anti-IgA and/or anti-IgG antibody and the binding between the ATA IgA and/or ATA IgG and the isolated tissue transglutaminase protein. Thus, if endogenous anti-tissue transglutaminase antibody (autoantibody) is present in the sample (e.g., a whole blood sample), it would increase the FRET signal (FIG. 10A). Therefore, in the presence of the endogenous anti-tissue transglutaminase antibody in the sample (e.g., a whole blood sample), the FRET signal is generated, as the fluorophore-labeled anti-IgA and/or anti-IgG antibody and the fluorophore-labeled isolated tissue transglutaminase protein are brought to proximity of each other. In some embodiments, the measurement of the presence or level of endogenous anti-tissue transglutaminase antibody in a sample (e.g., a whole blood sample) by TR-FRET may be used as an aid to determine subject's disease condition.

In another aspect of the disclosure, the disclosure provides an assay method that can detect, as well as distinguish, the presence or amount of anti-transglutaminase autoantibody (ATA) immunoglobulin A (IgA) and/or ATA immunoglobulin G (IgG) in a sample (FIG. 10A), the method comprising:

contacting the sample with a complex comprising an anti-IgA and/or anti-IgG antibody labeled with a donor fluorophore (or an acceptor fluorophore) and an isolated tissue transglutaminase labeled with an acceptor fluorophore (or a donor fluorophore);

incubating the sample with the complex for a time sufficient for the anti-IgA and/or anti-IgG antibody and the isolated tissue transglutaminase to bind to the ATA IgA and/or ATA IgG in the sample; and

exciting the sample using a light source to detect fluorescence emission signals associated with FRET,

wherein a detection of a fluorescence signal emitted by the acceptor fluorophore indicates the presence or amount of ATA IgA and/or ATA IgG.

In certain aspects, the sample is a biological sample. Suitable biological samples include, but are not limited to, whole blood, saliva, urine, a fecal specimen, plasma, tissue, biopsy, or serum. In a preferred aspect, the biological sample is whole blood or serum.

In certain aspects, the FRET energy donor compound is a cryptate, such as a lanthanide cryptate.

In certain aspects, the cryptate has an absorption wavelength between about 300 nm to about 400 nm such as about 325 nm to about 375 nm. In certain aspects, as shown in FIG. 15, cyptate dyes (Lumi4-Tb in FIG. 15) have four fluorescence emission peaks at about 490 nm, about 548 nm, about 587 nm, and 621 nm. Thus, as a donor, the cryptate is compatible with fluorescein-like (green zone) and Cy5 or DY-647-like (red zone) acceptor (e.g., green acceptor, NIR acceptor, or orange acceptor in FIG. 15) to perform TR-FRET experiments.

In certain aspects, the introduction of a time delay between a flash excitation and the measurement of the fluorescence at the acceptor emission wavelength allows to discriminate long lived from short-lived fluorescence and to increase signal-to-noise ratio.

Cryptates as FRET Donors

In certain aspects, the terbium cryptate molecule “Lumi4-Tb” from Lumiphore, marketed by Cisbio bioassays is used as the cryptate donor. The terbium cryptate “Lumi4-Tb” having the formula below, which can be coupled to an antibody by a reactive group, in this case, for example, an NHS ester:

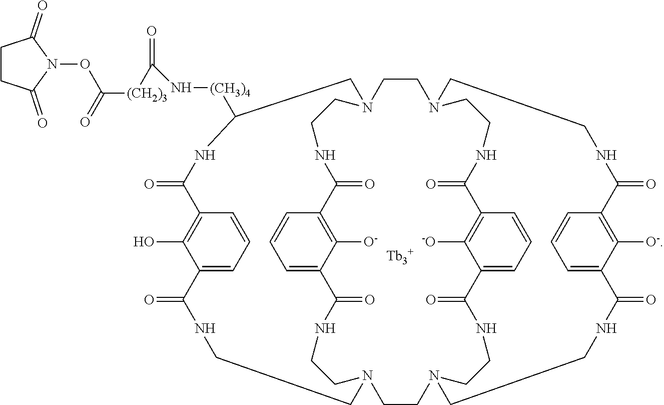

An activated ester (an NHS ester) can react with a primary amine on an antibody to make a stable amide bond. A maleimide on the cryptate and a thiol on the antibody can react together and make a thioether. Alkyl halidels react with amines and thiols to make alkylamines and thioethers, respectively. Any derivative providing a reactive moiety that can be conjugated to a antibody can be utilized herein. For example, in some embodiments, when an anti-human serum albumin antibody is used, the maleimide on the cryptate can react with a thiol on the antibody. In some embodiments, when a vitamin D-binding agent is used, the maleimide on the cryptate can react with a thiol on the antibody. In some embodiments, when an anti-C-reactive protein antibody is used, the maleimide on the cryptate can react with a thiol on the antibody. In some embodiments, when an anti-tissue transglutaminase antibody is used, the maleimide on the cryptate can react with a thiol on the antibody.

In certain other aspects, cryptates disclosed in WO2015157057, titled “Macrocycles” are suitable for use in the present disclosure. This publication contains cryptate molecules useful for labeling biomolecules. As disclosed therein, certain of the cryptates have the structure:

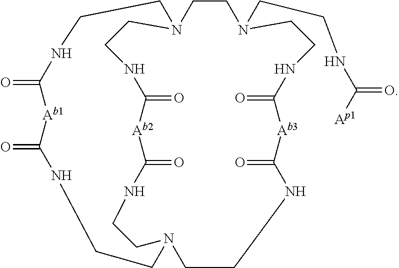

In certain other aspects, a terbium cryptate useful in the present disclosure is shown below:

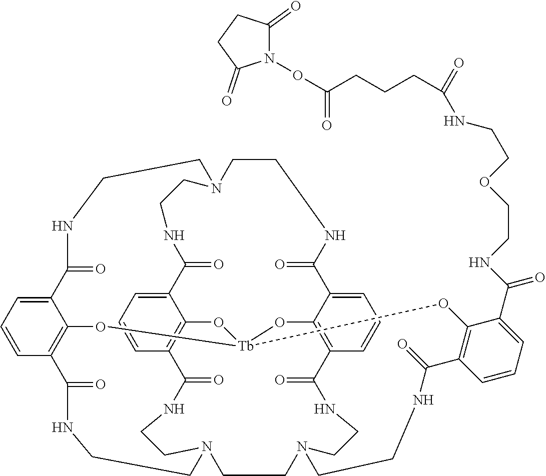

In certain aspects, the cryptates that are useful in the present invention are disclosed in WO 2018/130988, published Jul. 19, 2018. As disclosed therein, the compounds of Formula I are useful as FRET donors in the present disclosure:

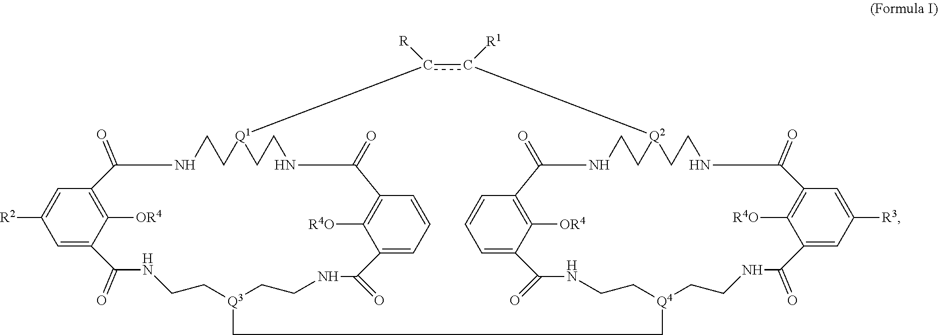

wherein when the dotted line is present, R and R¹ are each independently selected from the group consisting of hydrogen, halogen, hydroxyl, alkyl optionally substituted with one or more halogen atoms, carboxyl, alkoxycarbonyl, amido, sulfonato, alkoxycarbonylalkyl or alkylcarbonylalkoxy or alternatively, R and R¹ join to form an optionally substituted cyclopropyl group wherein the dotted bond is absent;
R² and R³ are each independently a member selected from the group consisting of hydrogen, halogen, SO₃H, —SO₂—X, wherein X is a halogen, optionally substituted alkyl, optionally substituted aryl, optionally substituted alkenyl, optionally substituted alkynyl, optionally substituted cycloalkyl, or an activated group that can be linked to a biomolecule, wherein the activated group is a member selected from the group consisting of a halogen, an activated ester, an activated acyl, optionally substituted alkylsulfonate ester, optionally substituted arylsulfonate ester, amino, formyl, glycidyl, halo, haloacetamidyl, haloalkyl, hydrazinyl, imido ester, isocyanato, isothiocyanato, maleimidyl, mercapto, alkynyl, hydroxyl, alkoxy, amino, cyano, carboxyl, alkoxycarbonyl, amido, sulfonato, alkoxycarbonylalkyl, cyclic anhydride, alkoxyalkyl, a water solubilizing group or L;
R⁴ are each independently a hydrogen, C₁-C₆ alkyl, or alternatively, 3 of the R⁴ groups are absent and the resulting oxides are chelated to a lanthanide cation; and
Q¹-Q⁴ are each independently a member selected from the group of carbon or nitrogen.

FRET Acceptors

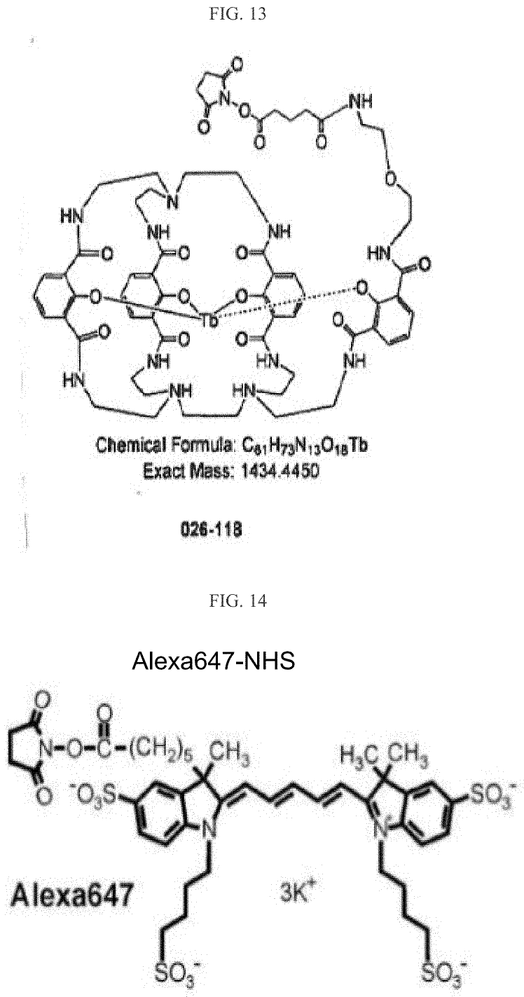

In order to detect a FRET signal, a FRET acceptor is required. The FRET acceptor has an excitation wavelength that overlaps with an emission wavelength of the FRET donor. In the present disclosure, a FRET signal of the acceptor is detected when an anti-human serum albumin antibody labeled with a donor fluorophore (or an acceptor fluorophore) binds to an isolated human serum albumin labeled with an acceptor fluorophore (or a donor fluorophore). A known amount of calibrators, i.e., standard curves (FIG. 2), can be used to interpolate the concentration levels of human serum albumin in a sample. As described above, the cryptate donor (FIG. 13) can be used to label the anti-human serum albumin antibody. Lumi4-Tb has 3 spectrally distinct peaks, at 490, 550 and 620 nm, which can be used for energy transfer (FIG. 15). Subsequently, a first acceptor can be used to label an anti-human serum albumin antibody.

Also, in the present disclosure, a FRET signal of the acceptor is detected when a vitamin D-binding agent labeled with a donor fluorophore (or an acceptor fluorophore) binds to an isolated vitamin D labeled with an acceptor fluorophore (or a donor fluorophore). A known amount of calibrators, i.e., standard curves (FIG. 5), can be used to interpolate the concentration levels of vitamin D in a sample. As described above, the cryptate donor (FIG. 13) can be used to label the vitamin D-binding agent. Lumi4-Tb has 3 spectrally distinct peaks, at 490, 550 and 620 nm, which can be used for energy transfer (FIG. 15). Subsequently, a first acceptor can be used to label a vitamin D-binding agent.

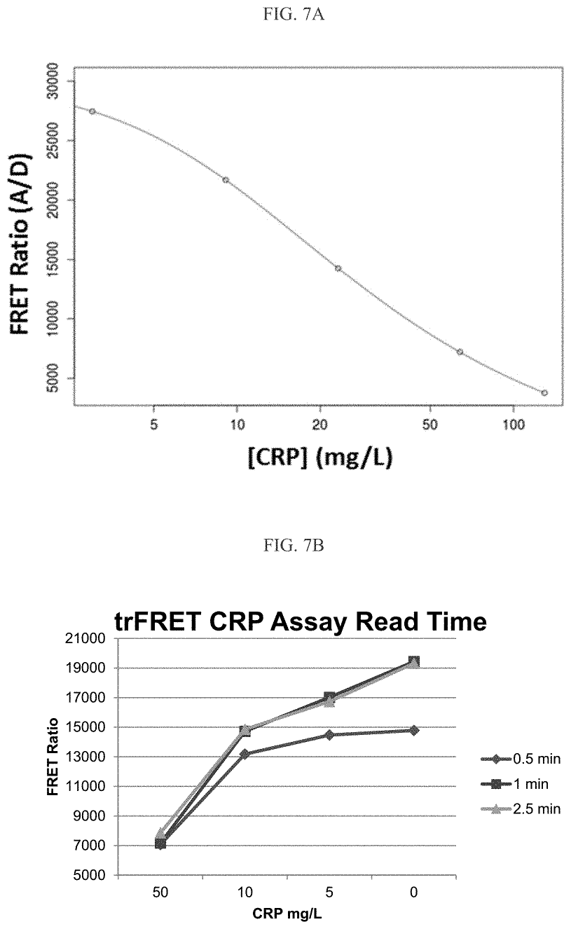

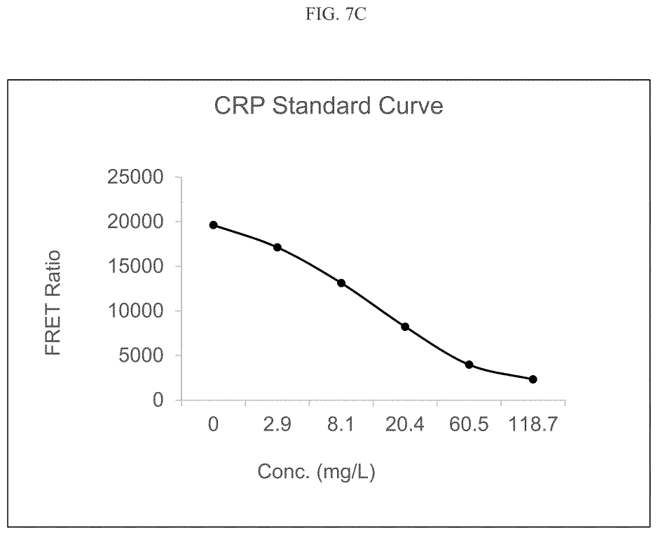

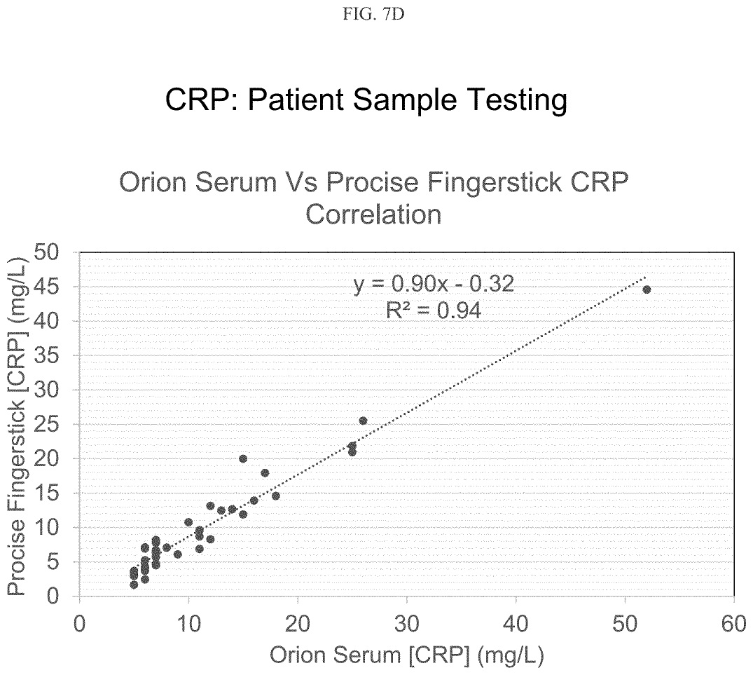

Also, in the present disclosure, a FRET signal of the acceptor is detected when an anti-C-reactive protein antibody labeled with a donor fluorophore (or an acceptor fluorophore) binds to an isolated C-reactive protein labeled with an acceptor fluorophore (or a donor fluorophore). A known amount of calibrators, i.e., standard curve (FIG. 7A), can be used to interpolate the concentration levels of C-reactive protein in a sample. As described above, the cryptate donor (FIG. 13) can be used to label the anti-C-reactive protein antibody. Lumi4 has 4 spectrally distinct peaks, at about 490 nm, about 545 nm, about 580 nm, and about 620 nm, which can be used for energy transfer (FIG. 15). Subsequently, a first acceptor can be used to label an anti-C-reactive protein antibody.

Also, in the present disclosure, a FRET signal of the acceptor is detected when an anti-tissue transglutaminase antibody labeled with a donor fluorophore (or an acceptor fluorophore) binds to an isolated tissue transglutaminase protein labeled with an acceptor fluorophore (or a donor fluorophore). A known amount of calibrators, i.e., standard curve (FIG. 9), can be used to interpolate the concentration levels of endogenous anti-tissue transglutaminase antibody in a sample. As described above, the cryptate donor (FIG. 13) can be used to label the anti-tissue transglutaminase antibody. Lumi4-Tb has 4 spectrally distinct peaks, at about 490 nm, about 548 nm, about 587 nm, and 621 nm, which can be used for energy transfer (FIG. 15). Subsequently, an acceptor can be used to label an anti-tissue transglutaminase antibody.

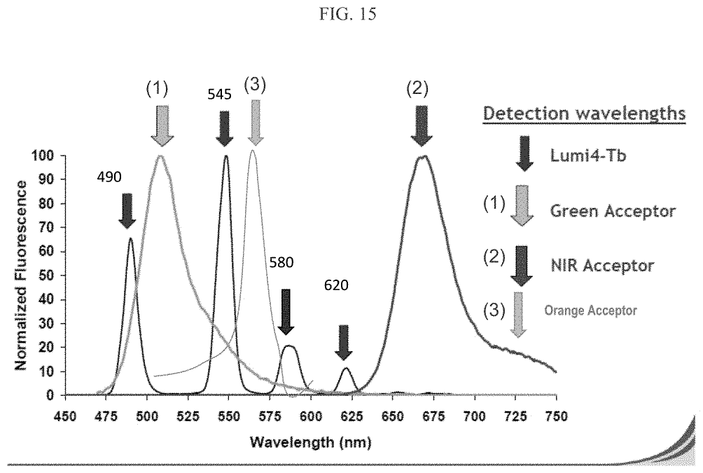

The acceptor molecules that can be used include, but are not limited to, fluorescein-like (green zone), Cy5, DY-647, Alexa Fluor 488, Alexa Fluor 546, Alexa Fluor 647 (FIG. 14), allophycocyanin (APC), and phycoerythrin (PE). Donor and acceptor fluorophores can be conjugated using a primary amine on an antibody.

Other acceptors include, but are not limited to, cyanin derivatives, D2, CYS, fluorescein, coumarin, rhodamine, carbopyronine, oxazine and its analogs, Alexa Fluor fluorophores, Crystal violet, perylene bisimide fluorophores, squaraine fluorophores, boron dipyrromethene derivatives, NBD (nitrobenzoxadiazole) and its derivatives, and DABCYL (4-((4-(dimethylamino)phenyl)azo)benzoic acid).

In one aspect, fluorescence can be characterized by wavelength, intensity, lifetime, and polarization.

Anti-Human Serum Albumin Antibodies

In one aspect, an anti-human serum albumin antibody (e.g., Catalog #ab10241 (Abcam), and shown to be specific for human serum albumin) can be used to conjugate to a donor fluorophore (e.g., cryptate) or an acceptor fluorophore. Other commercial anti-human serum albumin antibodies are available in the art, such as Catalog #15C7 (Biocompare) and Catalog #MIH3005 (ThermoFisher).

The methods herein for detecting the presence or levels of human serum albumin can use a variety of samples, which include a tissue sample, blood, biopsy, serum, plasma, saliva, urine, or stool sample.

Vitamin D-Binding Agents

Vitamin D-binding agents are proteins or molecules that can bind to vitamin D, such as anti-vitamin D antibodies or enzymes that can bind to vitamin D. For example, a radioimmunoassay (RIA), e.g., an RIA kit developed by DiaSorin S.p.A (Saluggia, Italy), uses an antibody with specificity for 25-hydroxy vitamin D (i.e., the sum of vitamin D2 and vitamin D3) after 25-hydroxy vitamin D is extracted from serum or plasma. In another example, chemiluminescent immunoassays (CLIA) also use an antibody to bind to the extracted vitamin D. In another example, enzyme-coupled vitamin D binding proteins, such as the ones used in enzyme immunoassays (e.g., enzyme immunoassay developed by Diazyme Laboratories), can also be used as vitamin D-binding agents in methods described herein. Other agents that can be used as vitamin D-binding agents are described in, e.g., Arneson and Arneson, Laboratory Medicine, 44:e38, 2013.

In one aspect, a vitamin D-binding agent is an anti-vitamin D antibody (e.g., Catalog #13-1080 (American Research Products), Catalog #A1090.2 (Immundiagnostik AG), Catalog #10-2256 (Fitzgerald Industries International), Catalog #HM674 (EastCoast Bio), and Catalog #abx100427 (Abbexa Ltd)) can be used to conjugate to a donor fluorophore (e.g., cryptate) or an acceptor fluorophore. Other commercial anti-vitamin D antibodies are available in the art.

The methods herein for detecting the presence or levels of vitamin D can use a variety of samples, which include a tissue sample, blood, biopsy, serum, plasma, saliva, urine, or stool sample.

Anti-C-Reactive Protein Antibodies

In one aspect, an anti-C-reactive protein antibody (e.g., Catalog #ab31156 (Abcam), and shown to be specific for C-reactive protein) can be used to conjugate to a donor fluorophore (e.g., cryptate) or an acceptor fluorophore. Other commercial anti-C-reactive protein antibodies are available in the art, such as Catalog #ab32412 (Biocompare) and Catalog #MAB17071 (R&D Systems).

The methods herein for detecting the presence or levels of C-reactive protein can use a variety of samples, which include a tissue sample, blood, biopsy, serum, plasma, saliva, urine, or stool sample.

Production of Antibodies

The generation and selection of antibodies not already commercially available can be accomplished several ways. For example, one way is to express and/or purify a polypeptide of interest (i.e., antigen) using protein expression and purification methods known in the art, while another way is to synthesize the polypeptide of interest using solid phase peptide synthesis methods known in the art. See, e.g., Guide to Protein Purification, Murray P. Deutcher, ed., Meth. Enzymol., Vol. 182 (1990); Solid Phase Peptide Synthesis, Greg B. Fields, ed., Meth. Enzymol., Vol. 289 (1997); Kiso et al., Chem. Pharm. Bull., 38:1192-99 (1990); Mostafavi et al., Biomed. Pept. Proteins Nucleic Acids, 1:255-60, (1995); and Fujiwara et al., Chem. Pharm. Bull., 44:1326-31 (1996). The purified or synthesized polypeptide can then be injected, for example, into mice or rabbits, to generate polyclonal or monoclonal antibodies. One skilled in the art will recognize that many procedures are available for the production of antibodies, for example, as described in Antibodies, A Laboratory Manual, Harlow and Lane, Eds., Cold Spring Harbor Laboratory, Cold Spring Harbor, N.Y. (1988). One skilled in the art will also appreciate that binding fragments or Fab fragments which mimic antibodies can also be prepared from genetic information by various procedures (see, e.g., Antibody Engineering: A Practical Approach, Borrebaeck, Ed., Oxford University Press, Oxford (1995); and Huse et al., J. Immunol., 149:3914-3920 (1992)).

In addition, numerous publications have reported the use of phage display technology to produce and screen libraries of polypeptides for binding to a selected target antigen (see, e.g, Cwirla et al., Proc. Natl. Acad. Sci. USA, 87:6378-6382 (1990); Devlin et al., Science, 249:404-406 (1990); Scott et al., Science, 249:386-388 (1990); and Ladner et al., U.S. Pat. No. 5,571,698). A basic concept of phage display methods is the establishment of a physical association between a polypeptide encoded by the phage DNA and a target antigen. This physical association is provided by the phage particle, which displays a polypeptide as part of a capsid enclosing the phage genome which encodes the polypeptide. The establishment of a physical association between polypeptides and their genetic material allows simultaneous mass screening of very large numbers of phage bearing different polypeptides. Phage displaying a polypeptide with affinity to a target antigen bind to the target antigen and these phage are enriched by affinity screening to the target antigen. The identity of polypeptides displayed from these phage can be determined from their respective genomes. Using these methods, a polypeptide identified as having a binding affinity for a desired target antigen can then be synthesized in bulk by conventional means (see, e.g., U.S. Pat. No. 6,057,098).

The antibodies that are generated by these methods can then be selected by first screening for affinity and specificity with the purified polypeptide antigen of interest and, if required, comparing the results to the affinity and specificity of the antibodies with other polypeptide antigens that are desired to be excluded from binding. The screening procedure can involve immobilization of the purified polypeptide antigens in separate wells of microtiter plates. The solution containing a potential antibody or group of antibodies is then placed into the respective microtiter wells and incubated for about 30 minutes to 2 hours. The microtiter wells are then washed and a labeled secondary antibody (e.g., an anti-mouse antibody conjugated to alkaline phosphatase if the raised antibodies are mouse antibodies) is added to the wells and incubated for about 30 minutes and then washed. Substrate is added to the wells and a color reaction will appear where antibody to the immobilized polypeptide antigen is present.

The antibodies so identified can then be further analyzed for affinity and specificity. In the development of immunoassays for a target protein (human serum albumin, vitamin D, C-reactive protein, or ATA), the purified target protein acts as a standard with which to judge the sensitivity and specificity of the immunoassay using the antibodies that have been selected. Because the binding affinity of various antibodies may differ, e.g., certain antibody combinations may interfere with one another sterically, assay performance of an antibody may be a more important measure than absolute affinity and specificity of that antibody.

Those skilled in the art will recognize that many approaches can be taken in producing antibodies or binding fragments and screening and selecting for affinity and specificity for the various polypeptides of interest, but these approaches do not change the scope of the present invention.

A. Polyclonal Antibodies

Polyclonal antibodies are preferably raised in animals by multiple subcutaneous (sc) or intraperitoneal (ip) injections of a polypeptide of interest and an adjuvant. It may be useful to conjugate the polypeptide of interest to a protein carrier that is immunogenic in the species to be immunized, such as, e.g., keyhole limpet hemocyanin, bovine thyroglobulin, or soybean trypsin inhibitor using a bifunctional or derivatizing agent. Non-limiting examples of bifunctional or derivatizing agents include maleimidobenzoyl sulfosuccinimide ester (conjugation through cysteine residues), N-hydroxysuccinimide (conjugation through lysine residues), glutaraldehyde, succinic anhydride, SOCl₂, and R₁N═C═NR, wherein R and R₁ are different alkyl groups.

Animals are immunized against the polypeptide of interest or an immunogenic conjugate or derivative thereof by combining, e.g., 100 μg (for rabbits) or 5 μg (for mice) of the antigen or conjugate with 3 volumes of Freund's complete adjuvant and injecting the solution intradermally at multiple sites. One month later, the animals are boosted with about ⅕ to 1/10 the original amount of polypeptide or conjugate in Freund's incomplete adjuvant by subcutaneous injection at multiple sites. Seven to fourteen days later, the animals are bled and the serum is assayed for antibody titer. Animals are typically boosted until the titer plateaus. Preferably, the animal is boosted with the conjugate of the same polypeptide, but conjugation to a different immunogenic protein and/or through a different cross-linking reagent may be used. Conjugates can also be made in recombinant cell culture as fusion proteins. In certain instances, aggregating agents such as alum can be used to enhance the immune response.

B. Monoclonal Antibodies

Monoclonal antibodies are generally obtained from a population of substantially homogeneous antibodies, i.e., the individual antibodies comprising the population are identical except for possible naturally-occurring mutations that may be present in minor amounts. Thus, the modifier “monoclonal” indicates the character of the antibody as not being a mixture of discrete antibodies. For example, monoclonal antibodies can be made using the hybridoma method described by Kohler et al., Nature, 256:495 (1975) or by any recombinant DNA method known in the art (see, e.g., U.S. Pat. No. 4,816,567).

In the hybridoma method, a mouse or other appropriate host animal (e.g., hamster) is immunized as described above to elicit lymphocytes that produce or are capable of producing antibodies which specifically bind to the polypeptide of interest used for immunization. Alternatively, lymphocytes are immunized in vitro. The immunized lymphocytes are then fused with myeloma cells using a suitable fusing agent, such as polyethylene glycol, to form hybridoma cells (see, e.g., Goding, Monoclonal Antibodies: Principles and Practice, Academic Press, pp. 59-103 (1986)). The hybridoma cells thus prepared are seeded and grown in a suitable culture medium that preferably contains one or more substances which inhibit the growth or survival of the unfused, parental myeloma cells. For example, if the parental myeloma cells lack the enzyme hypoxanthine guanine phosphoribosyl transferase (HGPRT), the culture medium for the hybridoma cells will typically include hypoxanthine, aminopterin, and thymidine (HAT medium), which prevent the growth of HGPRT-deficient cells.

Preferred myeloma cells are those that fuse efficiently, support stable high-level production of antibody by the selected antibody-producing cells, and/or are sensitive to a medium such as HAT medium. Examples of such preferred myeloma cell lines for the production of human monoclonal antibodies include, but are not limited to, murine myeloma lines such as those derived from MOPC-21 and MPC-11 mouse tumors (available from the Salk Institute Cell Distribution Center; San Diego, Calif.), SP-2 or X63-Ag8-653 cells (available from the American Type Culture Collection; Rockville, Md.), and human myeloma or mouse-human heteromyeloma cell lines (see, e.g., Kozbor, J. Immunol., 133:3001 (1984); and Brodeur et al., Monoclonal Antibody Production Techniques and Applications, Marcel Dekker, Inc., New York, pp. 51-63 (1987)).

The culture medium in which hybridoma cells are growing can be assayed for the production of monoclonal antibodies directed against the polypeptide of interest. Preferably, the binding specificity of monoclonal antibodies produced by hybridoma cells is determined by immunoprecipitation or by an in vitro binding assay, such as a radioimmunoassay (RIA) or an enzyme-linked immunoabsorbent assay (ELISA). The binding affinity of monoclonal antibodies can be determined using, e.g., the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (see, e.g., Goding, Monoclonal Antibodies: Principles and Practice, Academic Press, pp. 59-103 (1986)). Suitable culture media for this purpose include, for example, D-MEM or RPMI-1640 medium. In addition, the hybridoma cells may be grown in vivo as ascites tumors in an animal. The monoclonal antibodies secreted by the subclones can be separated from the culture medium, ascites fluid, or serum by conventional antibody purification procedures such as, for example, protein A-Sepharose, hydroxylapatite chromatography, gel electrophoresis, dialysis, or affinity chromatography.

DNA encoding the monoclonal antibodies can be readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody, to induce the synthesis of monoclonal antibodies in the recombinant host cells. See, e.g., Skerra et al., Curr. Opin. Immunol., 5:256-262 (1993); and Pluckthun, Immunol Rev., 130:151-188 (1992). The DNA can also be modified, for example, by substituting the coding sequence for human heavy chain and light chain constant domains in place of the homologous murine sequences (see, e.g., U.S. Pat. No. 4,816,567; and Morrison et al., Proc. Natl. Acad. Sci. USA, 81:6851 (1984)), or by covalently joining to the immunoglobulin coding sequence all or part of the coding sequence for a non-immunoglobulin polypeptide.

In a further embodiment, monoclonal antibodies or antibody fragments can be isolated from antibody phage libraries generated using the techniques described in, for example, McCafferty et al., Nature, 348:552-554 (1990); Clackson et al., Nature, 352:624-628 (1991); and Marks et al., J. Mol. Biol., 222:581-597 (1991). The production of high affinity (nM range) human monoclonal antibodies by chain shuffling is described in Marks et al., BioTechnology, 10:779-783 (1992). The use of combinatorial infection and in vivo recombination as a strategy for constructing very large phage libraries is described in Waterhouse et al., Nuc. Acids Res., 21:2265-2266 (1993). Thus, these techniques are viable alternatives to traditional monoclonal antibody hybridoma methods for the generation of monoclonal antibodies. Human Antibodies

As an alternative to humanization, human antibodies can be generated. In some embodiments, transgenic animals (e.g., mice) can be produced that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immun., 7:33 (1993); and U.S. Pat. Nos. 5,591,669, 5,589,369, and 5,545,807.

Alternatively, phage display technology (see, e.g., McCafferty et al., Nature, 348:552-553 (1990)) can be used to produce human antibodies and antibody fragments in vitro, using immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats as described in, e.g., Johnson et al., Curr. Opin. Struct. Biol., 3:564-571 (1993). Several sources of V-gene segments can be used for phage display. See, e.g., Clackson et al., Nature, 352:624-628 (1991). A repertoire of V genes from unimmunized human donors can be constructed and antibodies to a diverse array of antigens (including self-antigens) can be isolated essentially following the techniques described in Marks et al., J. Mol. Biol., 222:581-597 (1991); Griffith et al., EMBO J., 12:725-734 (1993); and U.S. Pat. Nos. 5,565,332 and 5,573,905.

In certain instances, human antibodies can be generated by in vitro activated B cells as described in, e.g., U.S. Pat. Nos. 5,567,610 and 5,229,275.

C. Antibody Fragments

Various techniques have been developed for the production of antibody fragments. Traditionally, these fragments were derived via proteolytic digestion of intact antibodies (see, e.g., Morimoto et al., J. Biochem. Biophys. Meth., 24:107-117 (1992); and Brennan et al., Science, 229:81 (1985)). However, these fragments can now be produced directly using recombinant host cells. For example, the antibody fragments can be isolated from the antibody phage libraries discussed above. Alternatively, Fab′-SH fragments can be directly recovered from E. coli cells and chemically coupled to form F(ab′)2 fragments (see, e.g., Carter et al., BioTechnology, 10:163-167 (1992)). According to another approach, F(ab′)2 fragments can be isolated directly from recombinant host cell culture. Other techniques for the production of antibody fragments will be apparent to those skilled in the art. In other embodiments, the antibody of choice is a single chain Fv fragment (scFv). See, e.g., PCT Publication No. WO 93/16185; and U.S. Pat. Nos. 5,571,894 and 5,587,458. The antibody fragment may also be a linear antibody as described, e.g., in U.S. Pat. No. 5,641,870. Such linear antibody fragments may be monospecific or bispecific.

D. Antibody Purification

When using recombinant techniques, antibodies can be produced inside an isolated host cell, in the periplasmic space of a host cell, or directly secreted from a host cell into the medium. If the antibody is produced intracellularly, the particulate debris is first removed, for example, by centrifugation or ultrafiltration. Carter et al., BioTech., 10:163-167 (1992) describes a procedure for isolating antibodies which are secreted into the periplasmic space of E. coli. Briefly, cell paste is thawed in the presence of sodium acetate (pH 3.5), EDTA, and phenylmethylsulfonylfluoride (PMSF) for about 30 min. Cell debris can be removed by centrifugation. Where the antibody is secreted into the medium, supernatants from such expression systems are generally concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from cells can be purified using, for example, hydroxylapatite chromatography, gel electrophoresis, dialysis, and affinity chromatography. The suitability of protein A as an affinity ligand depends on the species and isotype of any immunoglobulin Fc domain that is present in the antibody. Protein A can be used to purify antibodies that are based on human γ1, γ2, or γ4 heavy chains (see, e.g., Lindmark et al., J. Immunol. Meth., 62:1-13 (1983)). Protein G is recommended for all mouse isotypes and for human γ3 (see, e.g., Guss et al., EMBO J., 5:1567-1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker; Phillipsburg, N.J.) is useful for purification. Other techniques for protein purification such as fractionation on an ion-exchange column, ethanol precipitation, reverse phase HPLC, chromatography on silica, chromatography on heparin SEPHAROSE™, chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25 M salt).

E. Bispecific Antibodies

Bispecific antibodies are antibodies that have binding specificities for at least two different epitopes. Bispecific antibodies can be prepared as full-length antibodies or antibody fragments (e.g., F(ab′)2 bispecific antibodies).

Methods for making bispecific antibodies are known in the art. Traditional production of full-length bispecific antibodies is based on the co-expression of two immunoglobulin heavy chain-light chain pairs, where the two chains have different specificities (see, e.g., Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule is usually performed by affinity chromatography. Similar procedures are disclosed in PCT Publication No. WO 93/08829 and Traunecker et al., EMBO J., 10:3655-3659 (1991).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy chain constant region (CH1) containing the site necessary for light chain binding present in at least one of the fusions. DNA encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains into one expression vector when the expression of at least two polypeptide chains in equal ratios results in high yields or when the ratios are of no particular significance.

In some embodiments of this approach, the bispecific antibodies are composed of a hybrid immunoglobulin heavy chain with a first binding specificity (e.g., a first binding specificity for an epitope in human serum albumin, vitamin D, C-reactive protein, or anti-tissue transglutaminase antibody) in one arm, and a hybrid immunoglobulin heavy chain-light chain with a second binding specificity in the other arm. This asymmetric structure facilitates the separation of the desired bispecific compound from unwanted immunoglobulin chain combinations, as the presence of an immunoglobulin light chain in only one half of the bispecific molecule provides for a facile way of separation. See, e.g., PCT Publication No. WO 94/04690 and Suresh et al., Meth. Enzymol., 121:210 (1986).

According to another approach described in U.S. Pat. No. 5,731,168, the interface between a pair of antibody molecules can be engineered to maximize the percentage of heterodimers which are recovered from recombinant cell culture. The preferred interface comprises at least a part of the CH3 domain of an antibody constant domain. In this method, one or more small amino acid side-chains from the interface of the first antibody molecule are replaced with larger side chains (e.g., tyrosine or tryptophan). Compensatory “cavities” of identical or similar size to the large side-chain(s) are created on the interface of the second antibody molecule by replacing large amino acid side-chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Heteroconjugate antibodies can be made using any convenient cross-linking method. Suitable cross-linking agents and techniques are well-known in the art, and are disclosed in, e.g., U.S. Pat. No. 4,676,980.

Suitable techniques for generating bispecific antibodies from antibody fragments are also known in the art. For example, bispecific antibodies can be prepared using chemical linkage. In certain instances, bispecific antibodies can be generated by a procedure in which intact antibodies are proteolytically cleaved to generate F(ab′)2 fragments (see, e.g., Brennan et al., Science, 229:81 (1985)). These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody.

In some embodiments, Fab′-SH fragments can be directly recovered from E. coli and chemically coupled to form bispecific antibodies. For example, a fully humanized bispecific antibody F(ab′)2 molecule can be produced by the methods described in Shalaby et al., J. Exp. Med., 175: 217-225 (1992). Each Fab′ fragment was separately secreted from E. coli and subjected to directed chemical coupling in vitro to form the bispecific antibody.

Various techniques for making and isolating bispecific antibody fragments directly from recombinant cell culture have also been described. For example, bispecific antibodies have been produced using leucine zippers. See, e.g., Kostelny et al., J. Immunol., 148:1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy chain variable domain (VH) connected to a light chain variable domain (VL) by a linker which is too short to allow pairing between the two domains on the same chain. Accordingly, the VH and VL domains of one fragment are forced to pair with the complementary VL and VH domains of another fragment, thereby forming two antigen binding sites. Another strategy for making bispecific antibody fragments by the use of single-chain Fv (sFv) dimers is described in Gruber et al., J. Immunol., 152:5368 (1994).

Antibodies with more than two valencies are also contemplated. For example, trispecific antibodies can be prepared. See, e.g., Tutt et al., J. Immunol., 147:60 (1991).

### III. Human Serum Albumin

Human serum albumin is a protein that is in the human blood plasma and synthesized in the liver as a proalbumin precursor protein. The N-terminal peptide of the proalbumin precursor protein is removed to generate proalbumin, which is released from the rough endoplasmic reticulum into the Golgi vesicles where it is cleaved again to produce the matured serum albumin. Human serum albumin transports hormone and fatty acids, buffers blood pH, sustains plasma colloid oncotic pressure, binds nitric oxide, and regulates inflammation, among many other functions. The gene for human serum albumin is located on chromosome 4 in locus 4q13.3 and mutations in this gene can result in anomalous proteins. The human serum albumin gene is 16,961 nucleotides long from the putative cap site to the first poly(A) addition site. It is split into 15 exons that are symmetrically placed within the 3 domains thought to have arisen by triplication of a single primordial domain. Human serum albumin has a molecular weight of approximately 66.5 kDa and a serum half-life of approximately 20 days. Human serum albumin, UniProt ID No. P02768, is SEQ ID NO: 1.

In certain aspects, the methods described herein are used to measure and/or detect human serum albumin. In certain aspects, the concentration or level of human serum albumin is measured. In certain aspects, the biological sample in which human serum albumin is measured is whole blood.

In certain aspects, the concentration of human serum albumin is about 3 g/L to about 500 g/L. In certain aspect, the concentration of human serum albumin is about 3 g/L, 10 g/L, 20 g/L, 30 g/L, 40 g/L, 50 g/L, 60 g/L, 70 g/L, 80 g/L, 90 g/L, 100 g/L, 110 g/L, 120 g/L, 130 g/L, 140 g/L, 150 g/L, 160 g/L, 170 g/L, 180 g/L, 190 g/L, 200 g/L, 210 g/L, 220 g/L, 230 g/L, 240 g/L, 250 g/L, 260 g/L, 270 g/L, 280 g/L, 290 g/L, 300 g/L, 310 g/L, 320 g/L, 330 g/L, 340 g/L, 350 g/L, 360 g/L, 370 g/L, 380 g/L, 390 g/L, 400 g/L, 410 g/L, 420 g/L, 430 g/L, 440 g/L, 450 g/L, 460 g/L, 470 g/L, 480 g/L, 490 g/L, or 500 g/L.

In certain aspects, the normal control concentration of human serum albumin or reference value is about 35 g/L to about 50 g/L. In certain aspect, the amount of human serum albumin is about 35 g/L, 37 g/L, 39 g/L, 41 g/L, 43 g/L, 45 g/L, 47 g/L, 49 g/L, or 50 g/L.

In certain aspects, the concentration of human serum albumin in the biological sample is deemed elevated when it is at least 10% to about 60% greater than the normal control concentration of human serum albumin. In certain aspects, the concentration of human serum albumin in the biological sample is deemed elevated when it is at least about 10%, 15%, 20%, 25%, 30%, 35%, 40%, 45%, 50%, 55%, and/or 60% greater than the normal control concentration of human serum albumin. In certain aspects, the concentration of human serum albumin in the biological sample is deemed elevated when it is at least 50 g/L (e.g., 60 g/L, 70 g/L, 80 g/L, 90 g/L, 100 g/L, 110 g/L, 120 g/L, 130 g/L, 140 g/L, 150 g/L, 160 g/L, 170 g/L, 180 g/L, 190 g/L, 200 g/L, 210 g/L, 220 g/L, 230 g/L, 240 g/L, 250 g/L, 260 g/L, 270 g/L, 280 g/L, 290 g/L, 300 g/L, 310 g/L, 320 g/L, 330 g/L, 340 g/L, 350 g/L, 360 g/L, 370 g/L, 380 g/L, 390 g/L, 400 g/L, 410 g/L, 420 g/L, 430 g/L, 440 g/L, 450 g/L, 460 g/L, 470 g/L, 480 g/L, 490 g/L, or 500 g/L).

In some embodiments, the concentration of human serum albumin in the biological sample is deemed elevated when it is at least 100 g/L (e.g., at least 110 g/L, 120 g/L, 130 g/L, 140 g/L, 150 g/L, 160 g/L, 170 g/L, 180 g/L, 190 g/L, 200 g/L, 210 g/L, 220 g/L, 230 g/L, 240 g/L, 250 g/L, 260 g/L, 270 g/L, 280 g/L, 290 g/L, 300 g/L, 310 g/L, 320 g/L, 330 g/L, 340 g/L, 350 g/L, 360 g/L, 370 g/L, 380 g/L, 390 g/L, 400 g/L, 410 g/L, 420 g/L, 430 g/L, 440 g/L, 450 g/L, 460 g/L, 470 g/L, 480 g/L, 490 g/L, or 500 g/L).

In some embodiments, the concentration of human serum albumin in the biological sample is deemed low when it is below 35 g/L (e.g., 32 g/L, 30 g/L, 28 g/L, 26 g/L, 24 g/L, 22 g/L, 20 g/L, 18 g/L, 16 g/L, 14 g/L, 12 g/L, 10 g/L, 8 g/L, 6 g/L, or 4 g/L).

In some embodiments, the concentration of human serum albumin in the biological sample is deemed low when it is below 20 g/L (e.g., 18 g/L, 16 g/L, 14 g/L, 12 g/L, 10 g/L, 8 g/L, 6 g/L, or 4 g/L).

In certain aspects, the methods herein can be used to discriminate between nonalcoholic fatty liver (NAFL) and nonalcoholic steatohepatitis (NASH), by measuring a quantity of human serum albumin contained in blood collected from a subject.

In certain aspects, the methods herein can be used to determine the presence of fibrosis such as hepatic fibrosis by measuring a quantity of human serum albumin contained in blood collected from a subject.

In certain aspects, the method herein can be used to determine a degree of progression of a symptom of nonalcoholic fatty liver disease (NAFLD), by measuring a quantity of human serum albumin contained in blood collected from a subject.

In certain aspects, the methods herein can be used to determine the degree of progression of a symptom of NAFLD, NAFL or NASH by monitoring the level of human serum albumin.

### IV. Vitamin D

Vitamin D is a group of fat-soluble secosteroids, the most important of which are vitamin D3 (also known as cholecalciferol) and vitamin D2 (also known as ergocalciferol). Cholecalciferol and ergocalciferol can be ingested from the diet and from supplements. Only a few foods contain vitamin D, such as fish, eggs, and fortified dairy products. A major natural source of the vitamin is the synthesis of cholecalciferol in the skin from cholesterol through a chemical reaction that is dependent on sun exposure (specifically UVB radiation). Vitamin D performs a number of functions, such as increasing intestinal absorption of calcium, magnesium, and phosphate, and promoting bone growth.

Vitamin D from the diet, or from skin synthesis, is biologically inactive. An enzyme must hydroxylate it to convert it to the active form. This is performed in the liver and in the kidneys. Cholecalciferol is converted in the liver to calcifediol (25-hydroxycholecalciferol); ergocalciferol is converted to 25-hydroxy ergocalciferol. These two vitamin D metabolites (also called 25-hydroxyvitamin D or 25(OH)D) are measured in serum to determine a person's vitamin D level. Calcifediol is further hydroxylated by the kidneys to form calcitriol (also known as 1,25-dihydroxycholecalciferol), the biologically active form of vitamin D. Calcitriol circulates as a hormone in the blood, having a major role regulating the concentration of calcium and phosphate, and promoting the healthy growth and remodeling of bone. Calcitriol also has other effects, including some on cell growth, neuromuscular and immune functions, and reduction of inflammation.

In certain aspects, the methods described herein are used to measure and/or detect vitamin D. In certain aspects, the concentration or level of vitamin D is measured. In certain aspects, the biological sample in which vitamin D is measured is whole blood.

In certain aspects, the concentration of vitamin D is about 2 ng/mL to about 500 ng/mL. In certain aspect, the concentration of vitamin D is about 2 ng/mL, 5 ng/mL, 10 ng/mL, 20 ng/mL, 30 ng/mL, 40 ng/mL, 50 ng/mL, 60 ng/mL, 70 ng/mL, 80 ng/mL, 90 ng/mL, 100 ng/mL, 110 ng/mL, 120 ng/mL, 130 ng/mL, 140 ng/mL, 150 ng/mL, 160 ng/mL, 170 ng/mL, 180 ng/mL, 190 ng/mL, 200 ng/mL, 210 ng/mL, 220 ng/mL, 230 ng/mL, 240 ng/mL, 250 ng/mL, 260 ng/mL, 270 ng/mL, 280 ng/mL, 290 ng/mL, 300 ng/mL, 310 ng/mL, 320 ng/mL, 330 ng/mL, 340 ng/mL, 350 ng/mL, 360 ng/mL, 370 ng/mL, 380 ng/mL, 390 ng/mL, 400 ng/mL, 410 ng/mL, 420 ng/mL, 430 ng/mL, 440 ng/mL, 450 ng/mL, 460 ng/mL, 470 ng/mL, 480 ng/mL, 490 ng/mL, or 500 ng/mL.

In certain aspects, the normal control concentration of vitamin D or reference value is about 20 ng/mL to about 50 ng/mL. In certain aspect, the amount of vitamin D is about 20 ng/mL, 23 ng/mL, 25 ng/mL, 27 ng/mL, 29 ng/mL, 31 ng/mL, 33 ng/mL, 35 ng/mL, 37 ng/mL, 39 ng/mL, 41 ng/mL, 43 ng/mL, 45 ng/mL, 47 ng/mL, 49 ng/mL, or 50 ng/mL.

In certain aspects, the concentration of vitamin D in the biological sample is deemed elevated when it is at least 10% to about 60% greater than the normal control concentration of vitamin D. In certain aspects, the concentration of vitamin D in the biological sample is deemed elevated when it is at least about 10%, 15%, 20%, 25%, 30%, 35%, 40%, 45%, 50%, 55%, and/or 60% greater than the normal control concentration of vitamin D. In certain aspects, the concentration of vitamin D in the biological sample is deemed elevated when it is at least 50 ng/mL (e.g., 60 ng/mL, 70 ng/mL, 80 ng/mL, 90 ng/mL, 100 ng/mL, 110 ng/mL, 120 ng/mL, 130 ng/mL, 140 ng/mL, 150 ng/mL, 160 ng/mL, 170 ng/mL, 180 ng/mL, 190 ng/mL, 200 ng/mL, 210 ng/mL, 220 ng/mL, 230 ng/mL, 240 ng/mL, 250 ng/mL, 260 ng/mL, 270 ng/mL, 280 ng/mL, 290 ng/mL, 300 ng/mL, 310 ng/mL, 320 ng/mL, 330 ng/mL, 340 ng/mL, 350 ng/mL, 360 ng/mL, 370 ng/mL, 380 ng/mL, 390 ng/mL, 400 ng/mL, 410 ng/mL, 420 ng/mL, 430 ng/mL, 440 ng/mL, 450 ng/mL, 460 ng/mL, 470 ng/mL, 480 ng/mL, 490 ng/mL, or 500 ng/mL).

In some embodiments, the concentration of vitamin D in the biological sample is deemed elevated when it is at least 100 ng/mL (e.g., at least 110 ng/mL, 120 ng/mL, 130 ng/mL, 140 ng/mL, 150 ng/mL, 160 ng/mL, 170 ng/mL, 180 ng/mL, 190 ng/mL, 200 ng/mL, 210 ng/mL, 220 ng/mL, 230 ng/mL, 240 ng/mL, 250 ng/mL, 260 ng/mL, 270 ng/mL, 280 ng/mL, 290 ng/mL, 300 ng/mL, 310 ng/mL, 320 ng/mL, 330 ng/mL, 340 ng/mL, 350 ng/mL, 360 ng/mL, 370 ng/mL, 380 ng/mL, 390 ng/mL, 400 ng/mL, 410 ng/mL, 420 ng/mL, 430 ng/mL, 440 ng/mL, 450 ng/mL, 460 ng/mL, 470 ng/mL, 480 ng/mL, 490 ng/mL, or 500 ng/mL).

In some embodiments, the concentration of vitamin D in the biological sample is deemed low when it is below 20 ng/mL (e.g., 18 ng/mL, 16 ng/mL, 14 ng/mL, 12 ng/mL, 10 ng/mL, 8 ng/mL, 6 ng/mL, or 4 ng/mL).

In some embodiments, the concentration of vitamin D in the biological sample is deemed low when it is below 10 ng/mL (e.g., 8 ng/mL, 6 ng/mL, or 4 ng/mL).

In certain aspects, the methods herein can be used to discriminate between nonalcoholic fatty liver (NAFL) and nonalcoholic steatohepatitis (NASH), by measuring a quantity of vitamin D contained in blood collected from a subject.

In certain aspects, the methods herein can be used to determine the presence of fibrosis such as hepatic fibrosis by measuring a quantity of vitamin D contained in blood collected from a subject.

In certain aspects, the method herein can be used to determine a degree of progression of a symptom of nonalcoholic fatty liver disease (NAFLD), by measuring a quantity of vitamin D contained in blood collected from a subject.

In certain aspects, the methods herein can be used to determine the degree of progression of a symptom of NAFLD, NAFL or NASH by monitoring the level of vitamin D.

### V. C-Reactive Protein

C-reactive protein is a pentameric protein found in the blood plasma, whose circulating concentrations rise in response to inflammation. The protein is synthesized by the liver in response to factors released by macrophages and fat cells (adipocytes). The C-reactive protein gene is located on chromosome 1 (1q23.2). Each monomer of its pentameric structure has 224 amino acids, and a molecular mass of 25,106 Da. In serum, it assembles into stable pentameric structure with a discoid shape.

C-reactive protein is an acute-phase protein of hepatic origin that increases following interleukin-6 (IL-6) secretion by macrophages and T cells. Other inflammatory mediators that can increase C-reactive protein level are TGF-β1 and TNF-α. IL-6 is produced by macrophages, as well as adipocytes, in response to a wide range of acute and chronic inflammatory conditions, such as bacterial, viral, or fungal infections, rheumatic and other inflammatory diseases, malignancy; and tissue injury and necrosis. These conditions cause release of IL-6 and other cytokines that trigger the synthesis of C-reactive protein and fibrinogen by the liver. C-reactive protein binds to lysophosphatidylcholine expressed on the surface of dead or dying cells (and some types of bacteria) in order to activate the complement system via C1q and promote phagocytosis by macrophages, which clears necrotic and apoptotic cells and bacteria.

In healthy adults, the normal concentration of C-reactive protein is generally below 3.0 mg/L, e.g., between 0.8 mg/L to 3.0 mg/L. When there is a stimulus, the C-reactive protein level can increase dramatically, e.g., at least 5-fold (e.g., at least 10-fold, 20-fold, 30-fold, 40-fold, 50-fold, 60-fold, 70-fold, 80-fold, 90-fold, 100-fold, 120-fold, 140-fold, 160-fold, 180-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1,000-fold) more than its normal level. The plasma half-life of C-reactive protein is about 19 hours, and is constant in all medical conditions. Therefore, the only factor that affects the blood C-reactive protein concentration is its production rate, which increases with inflammation, infection, trauma, necrosis, malignancy, and allergic reactions.

In certain aspects, the methods described herein are used to measure and/or detect C-reactive protein. In certain aspects, the concentration or level of C-reactive protein is measured. In certain aspects, the biological sample in which C-reactive protein is measured is whole blood.

In certain aspects, the normal control concentration of C-reactive protein or reference value is below 3 mg/L (e.g., 2.8 mg/L, 2.6 mg/L, 2.4 mg/L, 2.2 mg/L, 2 mg/L, 1.8 mg/L, 1.6 mg/L, 1.4 mg/L, 1.2 mg/L, 1 mg/L, 0.8 mg/L, 0.6 mg/L, 0.4 mg/L, or 0.2 mg/L).

In certain aspects, the concentration of C-reactive protein in the biological sample is deemed elevated when it is at least 10% to about 60% greater than the normal control concentration of C-reactive protein. In certain aspects, the concentration of C-reactive protein in the biological sample is deemed elevated when it is at least about 10%, 15%, 20%, 25%, 30%, 35%, 40%, 45%, 50%, 55%, and/or 60% greater than the normal control concentration of C-reactive protein. In some embodiments, the concentration of C-reactive protein in the biological sample is deemed elevated when it is at least 5-fold (e.g., at least 10-fold, 20-fold, 30-fold, 40-fold, 50-fold, 60-fold, 70-fold, 80-fold, 90-fold, 100-fold, 120-fold, 140-fold, 160-fold, 180-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1,000-fold) more than its normal level.

In certain aspects, the concentration of C-reactive protein in the biological sample is deemed elevated when it is at least 15 mg/L (e.g., at least 20 mg/L, 30 mg/L, 40 mg/L, 50 mg/L, 60 mg/L, 70 mg/L, 80 mg/L, 90 mg/L, 100 mg/L, 110 mg/L, 120 mg/L, 130 mg/L, 140 mg/L, 150 mg/L, 160 mg/L, 170 mg/L, 180 mg/L, 190 mg/L, or 200 mg/L). In certain aspects, the concentration of C-reactive protein in the biological sample is deemed elevated when it is at least 30 mg/L (e.g., at least 35 mg/L, 40 mg/L, 50 mg/L, 60 mg/L, 70 mg/L, 80 mg/L, 90 mg/L, 100 mg/L, 110 mg/L, 120 mg/L, 130 mg/L, 140 mg/L, 150 mg/L, 160 mg/L, 170 mg/L, 180 mg/L, 190 mg/L, or 200 mg/L).

In certain aspects, the methods herein can be used to discriminate between nonalcoholic fatty liver (NAFL) and nonalcoholic steatohepatitis (NASH), by measuring a quantity of C-reactive protein contained in blood collected from a subject; and determining that the subject is affected with or possibly affected with NASH in a case that the quantity of C-reactive protein is elevated or larger than a reference value.

In certain aspects, the methods herein can be used to determine the presence of fibrosis such as hepatic fibrosis by measuring a quantity of C-reactive protein contained in blood collected from a subject; and determining that the subject has or possibly has a symptom of hepatic fibrosis in a case that the quantity of C-reactive protein is elevated or larger than a reference value.

In certain aspects, the method herein can be used to determine a degree of progression of a symptom of nonalcoholic fatty liver disease (NAFLD), by measuring a quantity of C-reactive protein contained in blood collected from a subject if the quantity of C-reactive protein is larger than a reference value.

In certain aspects, the methods herein can be used to determine the degree of progression of a symptom of NAFLD, NAFL or NASH by monitoring the level of C-reactive protein. The larger the value is, it is determined that a subject has a possibly high degree of progression of a symptom of NAFLD, NAFL or NASH. Alternatively, it may also be determined that the application of the therapeutic drug is possibly effective in a case that the value after the application of the therapeutic drug is lower than the index value before the application.

### VI. Anti-Transglutaminase Antibody

Anti-transglutaminase antibody (ATA) are autoantibodies against the transglutaminase protein. ATA IgGs and ATA IgAs are ATAs classified according to immunoglobulin reactivity subclass (IgA, IgG). Anti-transglutaminase antibodies are often found in patients with several conditions, including celiac disease, juvenile diabetes, inflammatory bowel disease, and various forms of arthritis. In celiac disease, anti-transglutaminase antibodies are involved in the destruction of the villous extracellular matrix and target the destruction of intestinal villous epithelial cells by killer cells. Deposits of anti-transglutaminase antibodies in the intestinal epithelium can be used to predict celiac disease.

In certain aspects, the disclosure includes an assay for detecting anti-tTG autoantibodies in a sample, whereby the presence of said autoantibodies indicates a gluten-induced disease.

In certain aspects, the disclosure provides a test-kit useful in the methods disclosed. The test-kit comprises an anti-tissue transglutaminase antibody labeled with a donor fluorophore (or acceptor) and an isolated tissue transglutaminase labeled with an acceptor fluorophore (or donor), for assaying anti-tTG autoantibodies in a sample.

In certain aspects, the methods described herein are used to measure and/or detect ATA IgA and/or ATA IgG. In certain aspects, the concentration or level of ATA IgA and/or ATA IgG is measured. In certain aspects, the biological sample in which ATA IgA and/or ATA IgG is measured is whole blood.

In certain aspects, the control concentration of ATA IgA is between about 7 mg/dL to about 4,000 mg/dL (e.g., 7 mg/dL, 10 mg/dL, 50 mg/dL, 100 mg/dL, 200 mg/dL, 300 mg/dL, 400 mg/dL, 500 mg/dL, 600 mg/dL, 700 mg/dL, 800 mg/dL, 900 mg/dL, 1000 mg/dL, 1100 mg/dL, 1200 mg/dL, 1300 mg/dL, 1400 mg/dL, 1500 mg/dL, 1600 mg/dL, 1700 mg/dL, 1800 mg/dL, 1900 mg/dL, 2000 mg/dL, 2100 mg/dL, 2200 mg/dL, 2300 mg/dL, 2400 mg/dL, 2500 mg/dL, 2600 mg/dL, 2700 mg/dL, 2800 mg/dL, 2900 mg/dL, 3000 mg/dL, 3100 mg/dL, 3200 mg/dL, 3300 mg/dL, 3400 mg/dL, 3500 mg/dL, 3600 mg/dL, 3700 mg/dL, 3800 mg/dL, 3900 mg/dL, or 4000 mg/dL).

In certain aspects, the normal control concentration of ATA IgA or reference value is between about 70 mg/dL and about 400 mg/dL (e.g., 70 mg/dL, 80 mg/dL, 100 mg/dL, 120 mg/dL, 140 mg/dL, 160 mg/dL, 180 mg/dL, 200 mg/dL, 220 mg/dL, 240 mg/dL, 260 mg/dL, 280 mg/dL, 300 mg/dL, 320 mg/dL, 340 mg/dL, 360 mg/dL, 380 mg/dL, or 400 mg/dL).

In certain aspects, the concentration of ATA IgA in the biological sample is deemed elevated when it is at least 10% to about 60% greater than the normal control concentration of ATA IgA. In certain aspects, the concentration of ATA IgA in the biological sample is deemed elevated when it is at least about 10%, 15%, 20%, 25%, 30%, 35%, 40%, 45%, 50%, 55%, and/or 60% greater than the normal control concentration of ATA IgA. In some embodiments, the concentration of ATA IgA in the biological sample is deemed elevated when it is at least 5-fold (e.g., at least 10-fold, 20-fold, 30-fold, 40-fold, 50-fold, 60-fold, 70-fold, 80-fold, 90-fold, 100-fold, 120-fold, 140-fold, 160-fold, 180-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1,000-fold) more than its normal level.

In certain aspects, the concentration of ATA IgA in the biological sample is deemed elevated when it is at least above 400 mg/dL (e.g., at least 420 mg/dL, 440 mg/dL, 460 mg/dL, 480 mg/dL, 500 mg/dL, 520 mg/dL, 540 mg/dL, 560 mg/dL, 580 mg/dL, 600 mg/dL, 620 mg/dL, 640 mg/dL, 660 mg/dL, 680 mg/dL, 700 mg/dL, 720 mg/dL, 740 mg/dL, 760 mg/dL, 780 mg/dL, 800 mg/dL, 820 mg/dL, 840 mg/dL, 860 mg/dL, 880 mg/dL, 900 mg/dL, 920 mg/dL, 940 mg/dL, 960 mg/dL, 980 mg/dL, or 1000 mg/dL).

In certain aspects, the control concentration of ATA IgG is between about 20 mg/dL to about 4,000 mg/dL (e.g., 20 mg/dL, 50 mg/dL, 100 mg/dL, 200 mg/dL, 300 mg/dL, 400 mg/dL, 500 mg/dL, 600 mg/dL, 700 mg/dL, 800 mg/dL, 900 mg/dL, 1000 mg/dL, 1100 mg/dL, 1200 mg/dL, 1300 mg/dL, 1400 mg/dL, 1500 mg/dL, 1600 mg/dL, 1700 mg/dL, 1800 mg/dL, 1900 mg/dL, 2000 mg/dL, 2100 mg/dL, 2200 mg/dL, 2300 mg/dL, 2400 mg/dL, 2500 mg/dL, 2600 mg/dL, 2700 mg/dL, 2800 mg/dL, 2900 mg/dL, 3000 mg/dL, 3100 mg/dL, 3200 mg/dL, 3300 mg/dL, 3400 mg/dL, 3500 mg/dL, 3600 mg/dL, 3700 mg/dL, 3800 mg/dL, 3900 mg/dL, or 4000 mg/dL).

In certain aspects, the normal control concentration of ATA IgG or reference value is between about 200 mg/dL to about 400 mg/dL (e.g., 200 mg/dL, 220 mg/dL, 240 mg/dL, 260 mg/dL, 280 mg/dL, 300 mg/dL, 320 mg/dL, 340 mg/dL, 360 mg/dL, 380 mg/dL, or 400 mg/dL).

In certain aspects, the concentration of ATA IgG in the biological sample is deemed elevated when it is at least 10% to about 60% greater than the normal control concentration of ATA IgG. In certain aspects, the concentration of ATA IgG in the biological sample is deemed elevated when it is at least about 10%, 15%, 20%, 25%, 30%, 35%, 40%, 45%, 50%, 55%, and/or 60% greater than the normal control concentration of ATA IgG. In some embodiments, the concentration of ATA IgG in the biological sample is deemed elevated when it is at least 5-fold (e.g., at least 10-fold, 20-fold, 30-fold, 40-fold, 50-fold, 60-fold, 70-fold, 80-fold, 90-fold, 100-fold, 120-fold, 140-fold, 160-fold, 180-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1,000-fold) more than its normal level.

In certain aspects, the concentration of ATA IgG in the biological sample is deemed elevated when it is at least above 400 mg/dL (e.g., at least 420 mg/dL, 440 mg/dL, 460 mg/dL, 480 mg/dL, 500 mg/dL, 520 mg/dL, 540 mg/dL, 560 mg/dL, 580 mg/dL, 600 mg/dL, 620 mg/dL, 640 mg/dL, 660 mg/dL, 680 mg/dL, 700 mg/dL, 720 mg/dL, 740 mg/dL, 760 mg/dL, 780 mg/dL, 800 mg/dL, 820 mg/dL, 840 mg/dL, 860 mg/dL, 880 mg/dL, 900 mg/dL, 920 mg/dL, 940 mg/dL, 960 mg/dL, 980 mg/dL, or 1000 mg/dL).

In certain aspects, the methods herein can be used to discriminate between nonalcoholic fatty liver (NAFL) and nonalcoholic steatohepatitis (NASH), by measuring a quantity of ATA IgA and/or ATA IgG contained in blood collected from a subject; and determining that the subject is affected with or possibly affected with NASH in a case that the quantity of ATA IgA and/or ATA IgG is elevated or larger than a reference value.

In certain aspects, the methods herein can be used to determine the presence of fibrosis such as hepatic fibrosis by measuring a quantity of ATA IgA and/or ATA IgG contained in blood collected from a subject; and determining that the subject has or possibly has a symptom of hepatic fibrosis in a case that the quantity of ATA IgA and/or ATA IgG is elevated or larger than a reference value.

In certain aspects, the method herein can be used to determine a degree of progression of a symptom of nonalcoholic fatty liver disease (NAFLD), by measuring a quantity of ATA IgA and/or ATA IgG contained in blood collected from a subject if the quantity of ATA IgA and/or ATA IgG is larger than a reference value.

In certain aspects, the methods herein can be used to determine the degree of progression of a symptom of NAFLD, NAFL or NASH by monitoring the level of ATA IgA and/or ATA IgG. The larger the value is, it is determined that a subject has a possibly high degree of progression of a symptom of NAFLD, NAFL or NASH. Alternatively, it may also be determined that the application of the therapeutic drug is possibly effective in a case that the value after the application of the therapeutic drug is lower than the index value before the application.

### VII. Device

Various instruments and devices are suitable for use in the present disclosure. Many spectrophotometers have the capability to measure fluorescence. Fluorescence is the molecular absorption of light energy at one wavelength and its nearly instantaneous re-emission at another, longer wavelength. Some molecules fluoresce naturally, and others must be modified to fluoresce.

A fluorescence spectrophotometer or fluorometer, fluorospectrometer, or fluorescence spectrometer measures the fluorescent light emitted from a sample at different wavelengths, after illumination with light source such as a xenon flash lamp. Fluorometers can have different channels for measuring differently-colored fluorescent signals (that differ in their wavelengths), such as green and blue, or ultraviolet and blue, channels. In one aspect, a suitable device includes an ability to perform a time-resolved fluorescence resonance energy transfer (FRET) experiment.

Suitable fluorometers can hold samples in different ways, including cuvettes, capillaries, Petri dishes, and microplates. The assays described herein can be performed on any of these types of instruments. In certain aspects, the device has an optional microplate reader, allowing emission scans in up to 384-well plates. Others models suitable for use hold the sample in place using surface tension.

Time-resolved fluorescence (TRF) measurement is similar to fluorescence intensity measurement. One difference, however, is the timing of the excitation/measurement process. When measuring fluorescence intensity, the excitation and emission processes are simultaneous: the light emitted by the sample is measured while excitation is taking place. Even though emission systems are very efficient at removing excitation light before it reaches the detector, the amount of excitation light compared to emission light is such that fluorescent intensity measurements exhibit elevated background signals. The present disclosure offers a solution to this issue. Time resolve FRET relies on the use of specific fluorescent molecules that have the property of emitting over long periods of time (measured in milliseconds) after excitation, when most standard fluorescent dyes (e.g., fluorescein) emit within a few nanoseconds of being excited. As a result, it is possible to excite cryptate lanthanides using a pulsed light source (e.g., Xenon flash lamp or pulsed laser), and measure after the excitation pulse.

As the donor and acceptor fluorescent compounds attached to the antibodies move closer together, an energy transfer is caused from the donor compound to the acceptor compound, resulting in a decrease in the fluorescence signal emitted by the donor compound and an increase in the signal emitted by the acceptor compound, and vice-versa. The majority of biological phenomena involving interactions between different partners will therefore be able to be studied by measuring the change in FRET between two fluorescent compounds coupled with compounds which will be at a greater or lesser distance, depending on the biological phenomenon in question.

The FRET signal can be measured in different ways: measurement of the fluorescence emitted by the donor alone, by the acceptor alone or by the donor and the acceptor, or measurement of the variation in the polarization of the light emitted in the medium by the acceptor as a result of FRET. One can also include measurement of FRET by observing the variation in the lifetime of the donor, which is facilitated by using a donor with a long fluorescence lifetime, such as rare earth complexes (especially on simple equipment like plate readers). Furthermore, the FRET signal can be measured at a precise instant or at regular intervals, making it possible to study its change over time and thereby to investigate the kinetics of the biological process studied.

In certain aspects, the device disclosed in PCT/IB2019/051213, filed Feb. 14, 2019 is used, which is hereby incorporated by reference. That disclosure in that application generally relates to analyzers that can be used in point-of-care settings to measure the absorbance and fluorescence of a sample with minimal or no user handling or interaction. The disclosed analyzers provide advantageous features of more rapid and reliable analyses of samples having properties that can be detected with each of these two approaches. For example, it can be beneficial to quantify both the fluorescence and absorbance of a blood sample being subjected to a diagnostic assay. In some analytical workflows, the hematocrit of a blood sample can be quantified with an absorbance assay, while the signal intensities measured in a FRET assay can provide information regarding other components of the blood sample.

One apparatus disclosed in PCT/IB2019/051213 is useful for detecting an emission light from a sample, and absorbance of a transillumination light by the sample, which comprises a first light source configured to emit an excitation light having an excitation wavelength. The apparatus further comprises a second light source configured to transilluminate the sample with the transillumination light. The apparatus further comprises a first light detector configured to detect the excitation light, and a second light detector configured to detect the emission light and the transillumination light. The apparatus further comprises a dichroic mirror configured to (1) epi-illuminate the sample by reflecting at least a portion of the excitation light, (2) transmit at least a portion of the excitation light to the first light detector, (3) transmit at least a portion of the emission light to the second light detector, and (4) transmit at least a portion of the transillumination light to the second light detector.

One suitable cuvette for use in the present disclosure is disclosed in PCT/IB2019/051215, filed Feb. 14, 2019. One of the provided cuvettes comprises a hollow body enclosing an inner chamber having an open chamber top. The cuvette further comprises a lower lid having an inner wall, an outer wall, an open lid top, and an open lid bottom. At least a portion of the lower lid is configured to fit inside the inner chamber proximate to the open chamber top. The lower lid comprises one or more (e.g., two or more) containers connected to the inner wall, wherein each of the containers has an open container top. In certain aspects, the lower lid comprises two or more such containers. The lower lid further comprises a securing means connected to the hollow body. The cuvette further comprises an upper lid wherein at least a portion of the upper lid is configured to fit inside the lower lid proximate to the open lid top.

### VIII. Examples

### Example 1

This example illustrates a method of this disclosure detecting the presence and amounts of human serum albumin in a TR-FRET assay. As shown in FIG. 1, an isolated human serum albumin protein labeled with an acceptor fluorophore binds to an anti-human serum albumin antibody (MAB-1) labeled with a donor fluorophore. The human serum albumin analyte is in a sample from a patient (i.e., whole blood sample) and it binds to anti-human serum albumin antibody labeled with the donor fluorophore, thus, disrupting the FRET signal. In the presence of a high amount of human serum albumin, the FRET signal is low, since the human serum albumin in the sample (e.g., a whole blood sample) blocks or competes with the binding of the isolated human serum albumin to the anti-human serum albumin antibody. The decrease in FRET signal is proportional to the level of human serum albumin present in the patient's blood as interpolated from a known amount of human serum albumin calibrators (FIG. 2). Table 1 below shows the corresponding numerical data for FIG. 2.

TABLE 1

| Standards | Albumin [mg/dL] | % Delta F (Delta R/R0*100) |
|---|---|---|
| 1 | 10000 | 116 |
| 2 | 5000 | 133 |
| 3 | 2500 | 164 |
| 4 | 1250 | 212 |
| 5 | 625 | 298 |
| 6 | 313 | 460 |
| 7 | 156 | 698 |
| 8 | 78 | 1065 |
| 9 | 39 | 1557 |
| 10 | 20 | 1921 |
| 11 | 10 | 2352 |
| 12 | 0 | 3154 |

This example illustrates how the method of this disclosure for detecting the presence and amounts of human serum albumin (HAS) using a TR-FRET assay in the format shown in FIG. 1 performs when testing patient samples form a variety of conditions known to affect human serum albumin levels. The results from individual serum samples tested on both TR-FRET and Beckman Coulter IMMAGE® HSA assays are shown below in Table 2. Also included within Table 2 are the % CVs from the TR-FRET assay and the underlying disease associated with the patient sample.

TABLE 2

| | Beckman Coulter | TR-FRET | |
|---|---|---|---|
| | IMMAGE ® [HSA] | [HSA] | |
| Diagnosis ® | (mg/dL) | (mg/dL) | CV % |
| Normal | 4310 | 4152 | 10.4 |
| Normal | 4100 | 4470 | 8 |
| Normal | 4350 | 4566 | 8.5 |
| Normal | 3860 | 4000 | 7.7 |
| Normal | 4800 | 4941 | 6.1 |
| IBS - Constipation | 4250 | 3107 | 10.2 |
| IBS - Constipation | 3900 | 4175 | 10.6 |
| Functional Constipation | 3430 | 3503 | 7.4 |
| IBS - Mixed/ | 4210 | 4224 | 3.9 |
| Alternating | | | |
| IBS - Mixed/ | 3560 | 3605 | 6.5 |
| Alternating | | | |
| Functional Constipation | 4170 | 4377 | 5.1 |
| Functional Dyspepsia | 4470 | 4526 | 5.4 |
| Functional Constipation | 4140 | 4262 | 1.2 |
| IBS - Constipation | 3680 | 3744 | 5.2 |
| IBS - Constipation | 4640 | 4740 | 5 |
| IBS - Constipation | 4130 | 3978 | 7 |
| IBS - Mixed/ | 4290 | 4081 | 5.1 |
| Alternating | | | |
| IBS - Constipation | 4290 | 4092 | 2.7 |
| IBS - Constipation | 3940 | 3694 | 5.6 |
| IBS - Constipation | 3970 | 3774 | 3.5 |
| Normal | 4240 | 4262 | 4 |
| Normal | 4270 | 4092 | 4.5 |
| Normal | 4790 | 4339 | 4.3 |
| Normal | 4420 | 4069 | 3.9 |
| Normal | 4390 | 4237 | 8.5 |
| Normal | 4120 | 4069 | 7.1 |
| Normal | 4350 | 4637 | 10.1 |
| Normal | 4070 | 4140 | 9.4 |
| Normal | 3810 | 4011 | 9 |
| IBD - Crohn's Disease | 3460 | 3634 | 9.1 |
| IBS - Constipation | 4460 | 4457 | 6.4 |
| Normal | 3990 | 4364 | 5.2 |
| Normal | 4720 | 4468 | 1.6 |
| Normal | 4300 | 4196 | 5.3 |
| Normal | 4560 | 4723 | 3.5 |
| IBS - Mixed/ | 4990 | 5226 | 4.9 |
| Alternating | | | |
| IBS - Diarrhea | 4080 | 4171 | 1.5 |
| IBS - Constipation | 4700 | 4876 | 4.7 |
| Normal | 4190 | 4453 | 4 |
| Normal | 4640 | 4830 | 3.8 |
| Functional Constipation | 4380 | 4620 | 6.7 |
| Functional Constipation | 4090 | 4196 | 3.3 |
| Functional Constipation | 4230 | 4348 | 0.6 |
| Functional Constipation | 4220 | 4171 | 2.7 |
| Functional Constipation | 3870 | 4427 | 0.7 |
| Functional Dyspepsia | 4160 | 4427 | 3.5 |
| Functional Constipation | 3920 | 3800 | 3.3 |
| Functional Dyspepsia | 4770 | 4508 | 0.8 |
| Functional Dyspepsia | 4600 | 4440 | 4.2 |
| Functional Constipation | 4360 | 4564 | 1.4 |
| Functional Constipation | 4210 | 4400 | 6.5 |
| Functional Dyspepsia | 4710 | 4876 | 3.4 |
| Functional Dyspepsia | 4440 | 4753 | 4.5 |
| Functional Diarrhea | 4460 | 4564 | 6.3 |
| IBS - Mixed/ | 4620 | 4468 | 1.7 |
| Alternating | | | |
| Celiac Disease | 3100 | 3101 | 4.8 |
| Functional Constipation | 4310 | 3974 | 12.8 |
| IBS - Diarrhea | 3560 | 3611 | 6.7 |
| IBS - Diarrhea | 3990 | 4089 | 5.8 |
| Functional Constipation | 4330 | 3659 | 5 |
| Normal | 4310 | 4152 | 10.4 |
| Normal | 4100 | 4470 | 8 |
| Normal | 4350 | 4566 | 8.5 |
| Normal | 3860 | 4000 | 7.7 |
| Normal | 4800 | 4941 | 6.1 |
| IBS - Constipation | 4250 | 3107 | 10.2 |
| IBS - Constipation | 3900 | 4175 | 10.6 |
| Functional Constipation | 3430 | 3503 | 7.4 |
| IBS - Mixed/ | 4210 | 4224 | 3.9 |
| Alternating | | | |
| IBS - Mixed/ | 3560 | 3605 | 6.5 |
| Alternating | | | |
| Functional Constipation | 4170 | 4377 | 5.1 |
| Functional Dyspepsia | 4470 | 4526 | 5.4 |
| Functional Constipation | 4140 | 4262 | 1.2 |
| IBS - Constipation | 3680 | 3744 | 5.2 |
| IBS - Constipation | 4640 | 4740 | 5 |
| IBS - Constipation | 4130 | 3978 | 7 |
| IBS - Mixed/ | 4290 | 4081 | 5.1 |
| Alternating | | | |
| IBS - Constipation | 4290 | 4092 | 2.7 |
| IBS - Constipation | 3940 | 3694 | 5.6 |
| IBS - Constipation | 3970 | 3774 | 3.5 |
| Normal | 4240 | 4262 | 4 |
| Normal | 4270 | 4092 | 4.5 |
| Normal | 4790 | 4339 | 4.3 |
| Normal | 4420 | 4069 | 3.9 |
| Normal | 4390 | 4237 | 8.5 |
| Normal | 4120 | 4069 | 7.1 |
| Normal | 4350 | 4637 | 10.1 |
| Normal | 4070 | 4140 | 9.4 |
| Normal | 3810 | 4011 | 9 |
| IBD - Crohn's Disease | 3460 | 3634 | 9.1 |
| IBS - Constipation | 4460 | 4457 | 6.4 |
| Normal | 3990 | 4364 | 5.2 |
| Normal | 4720 | 4468 | 1.6 |
| Normal | 4300 | 4196 | 5.3 |
| Normal | 4560 | 4723 | 3.5 |
| IBS - Mixed/Alternating | 4990 | 5226 | 4.9 |
| IBS - Diarrhea | 4080 | 4171 | 1.5 |
| IBS - Constipation | 4700 | 4876 | 4.7 |
| Normal | 4190 | 4453 | 4 |
| Normal | 4640 | 4830 | 3.8 |
| Functional Constipation | 4380 | 4620 | 6.7 |
| Functional Constipation | 4090 | 4196 | 3.3 |
| Functional Constipation | 4230 | 4348 | 0.6 |
| Functional Constipation | 4220 | 4171 | 2.7 |
| Functional Constipation | 3870 | 4427 | 0.7 |
| Functional Dyspepsia | 4160 | 4427 | 3.5 |
| Functional Constipation | 3920 | 3800 | 3.3 |
| Functional Dyspepsia | 4770 | 4508 | 0.8 |
| Functional Dyspepsia | 4600 | 4440 | 4.2 |
| Functional Constipation | 4360 | 4564 | 1.4 |
| Functional Constipation | 4210 | 4400 | 6.5 |
| Functional Dyspepsia | 4710 | 4876 | 3.4 |
| Functional Dyspepsia | 4440 | 4753 | 4.5 |
| Functional Diarrhea | 4460 | 4564 | 6.3 |
| IBS - Mixed/ | 4620 | 4468 | 1.7 |
| Alternating | | | |
| Celiac Disease | 3100 | 3101 | 4.8 |
| Functional Constipation | 4310 | 3974 | 12.8 |
| IBS - Diarrhea | 3560 | 3611 | 6.7 |
| IBS - Diarrhea | 3990 | 4089 | 5.8 |
| Functional Constipation | 4330 | 3659 | 5 |

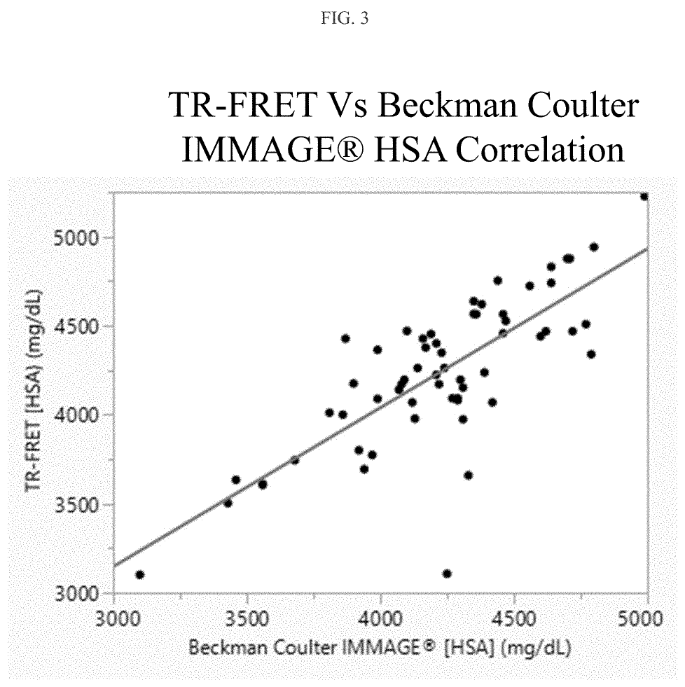

This example illustrates how the method of this disclosure for detecting the presence and amounts of human serum albumin (HAS) using a TR-FRET assay in the format shown in FIG. 1 correlates to the Beckman Coulter IMMAGE® HSA assay when testing patient samples form a variety of conditions known to affect human serum albumin levels. The results from individual serum samples from Table 2 are graphically displayed in FIG. 3.

Donor fluorophore, Lumi4-Tb (also called Tb-H22TRENIAM-5LIO-NHS, FIG. 13), can be used to label an anti-human serum albumin antibody. Lumi4-Tb has 3 spectrally distinct peaks, at 490, 550, and 620 nm, which can be used for energy transfer (FIG. 15). The acceptor fluorophores that can be used include but are not limited to: AlexaFluor 488, AlexaFluor 546, and AlexaFluor 647 (FIG. 15). Donor and acceptor fluorophores can be conjugated to antibodies using primary amines on antibodies.

The sequence of human serum albumin (UniProt ID NO. P02768) is shown below:

SEQ ID NO: 1:
MKWVTFISLLFLFSSAYSRGVFRRDAHKSEVAHRFKDLGEENFKALVLIAF
AQYLQQCPFEDHVKLVNEVTEFAKTCVADESAENCDKSLHTLFGDKLCTVA
TLRETYGEMADCCAKQEPERNECFLQHKDDNPNLPRLVRPEVDVMCTAFHD
NEETFLKKYLYEIARRHPYFYAPELLFFAKRYKAAFTECCQAADKAACLLP
KLDELRDEGKASSAKQRLKCASLQKFGERAFKAWAVARLSQRFPKAEFAEV
SKLVTDLTKVHTECCHGDLLECADDRADLAKYICENQDSISSKLKECCEKP
LLEKSHCIAEVENDEMPADLPSLAADFVESKDVCKNYAEAKDVFLGMFLYE
YARRHPDYSVVLLLRLAKTYETTLEKCCAAADPHECYAKVFDEFKPLVEEP
QNLIKQNCELFEQLGEYKFQNALLVRYTKKVPQVSTPTLVEVSRNLGKVGS
KCCKHPEAKRMPCAEDYLSVVLNQLCVLHEKTPVSDRVTKCCTESLVNRRP
CFSALEVDETYVPKEFNAETFTFHADICTLSEKERQIKKQTALVELVKHKP
KATKEQLKAVMDDFAAFVEKCCKADDKETCFAEEGKKLVAASQAALGL

### Example 2

This example illustrates a method of this disclosure detecting the presence and amounts of vitamin D in a TR-FRET assay. As shown in FIG. 4, an isolated vitamin D labeled with an acceptor fluorophore binds to a vitamin D-binding agent (e.g., an anti-vitamin D antibody (MAB-1)) labeled with a donor fluorophore. The vitamin D analyte is in a sample from a patient (i.e., whole blood sample) and it binds to anti-vitamin D antibody labeled with the donor fluorophore, thus, disrupting the FRET signal. In the presence of a high amount of vitamin D in the sample, the FRET signal is low, since the vitamin D in the sample (e.g., a whole blood sample) blocks or competes with the binding of the isolated vitamin D to the vitamin D-binding agent (e.g., an anti-vitamin D antibody (MAB-1)). The decrease in FRET signal is proportional to the level of vitamin D present in the patient's blood as interpolated from a known amount of vitamin D calibrators (FIG. 5 and Table 3).

TABLE 3

| Vitamin D | | | |
|---|---|---|---|
| Concentration | Average % | | |
| (ng/mL) | Delta F | Std Dev | % CV |
| 0.559 | 366.09 | 4.42 | 1.21 |
| 1.1 | 357.37 | 3.27 | 0.91 |
| 2.2 | 342.99 | 2.92 | 0.85 |
| 4.47 | 312.83 | 3.64 | 1.16 |
| 8.9 | 260.29 | 4.3 | 1.65 |
| 17.89 | 196.97 | 4.82 | 2.45 |
| 35.78 | 125.48 | 5.37 | 4.28 |
| 71.56 | 74.7 | 2.25 | 3.01 |
| 143 | 39.4 | 0.71 | 1.81 |
| 286 | 18.61 | 1.33 | 7.12 |
| 572 | 6.52 | 0.8 | 12 |
| 1145 | 0 | 1.88 | n/a |

Donor fluorophore, Lumi4-Tb (also called Tb-H22TRENIAM-5LIO-NHS, FIG. 13), can be used to label a vitamin D-binding agent (e.g., an anti-vitamin D antibody (MAB-1)). Lumi4-Tb has 3 spectrally distinct peaks, at 490, 550, and 620 nm, which can be used for energy transfer (FIG. 15). The acceptor fluorophores that can be used include but are not limited to: AlexaFluor 488, AlexaFluor 546, and AlexaFluor 647 (FIG. 15). Donor and acceptor fluorophores can be conjugated to antibodies using primary amines on antibodies.

### Example 3

This example illustrates a method of this disclosure detecting the presence and amounts of C-reactive protein in a TR-FRET assay. As shown in FIG. 6A, an isolated C-reactive protein (CRP) labeled with a donor fluorophore binds to an anti-C-reactive protein antibody (MAB-1) labeled with an acceptor fluorophore. The C-reactive protein analyte is in a sample from a patient (i.e., whole blood sample) and it binds to anti-C-reactive protein antibody labeled with the acceptor fluorophore, thus, disrupting the FRET signal. In the presence of a high amount of C-reactive protein, the FRET signal is low, since the C-reactive protein in the sample (e.g., a whole blood sample) blocks or competes with the binding of the isolated C-reactive protein to the anti-C-reactive protein antibody. The decrease in FRET signal is proportional to the level of C-reactive protein present in the patient's blood as interpolated from a known amount of C-reactive protein calibrators (FIG. 7A). FIG. 7C also shows a standard curve generated for C-reactive protein. Table 4 below shows the corresponding numerical data for FIG. 7C.

TABLE 4

| | Expected | | | | Calculated | | |
|---|---|---|---|---|---|---|---|
| | Conc. | | | | Conc. | % | % |
| Standards | (mg/L) | FRETRatio | DonorRFU | Acceptor1RFU | (mg/L) | CV | Recovery |
| CAL1_1 | 118.7 | 2325 | 128559 | 29884 | 119.6 | 0.02 | 100.7 |
| CAL2_1 | 60.5 | 3978 | 125048 | 49741 | 59.7 | 0.01 | 98.7 |
| CAL3_1 | 20.4 | 8224 | 125176 | 102937 | 20.6 | 0.02 | 101.0 |
| CAL4_1 | 8.1 | 13122 | 123445 | 161982 | 8.0 | 0.02 | 99.3 |
| CAL5_1 | 2.9 | 17117 | 122701 | 210025 | 2.9 | 0.05 | 100.6 |
| CAL6_1 | 0 | 19622 | 120818 | 237064 | 0.1 | | |

As shown in FIG. 6B, the reverse configuration works as well. In FIG. 6B, an isolated C-reactive protein (CRP) labeled with an acceptor fluorophore binds to an anti-C-reactive protein antibody (MAB-1) labeled with a donor fluorophore. The C-reactive protein analyte is in a sample from a patient (i.e., whole blood sample) and it binds to anti-C-reactive protein antibody labeled with the donor fluorophore, thus, disrupting the FRET signal. FIG. 6C is another schematic that shows that the FRET signal is inversely proportional to the concentration of CRP in a patient's sample.

This example illustrates how the method of this disclosure for detecting the presence and amounts of C-reactive protein (CRP) using a TR-FRET assay in the format shown in FIGS. 6A and 6B correlates to the Orion QuikRead go assay. For the correlation, matched pair samples from fingerstick collected whole blood, tested with TR-FRET, and serum, tested with Orion QuikRead go, are graphically displayed in FIG. 7D.

Donor fluorophore, Lumi4-Tb (also called Tb-H22TRENIAM-5LIO-NHS, FIG. 13), can be used to label an isolated C-reactive protein (CRP). Lumi4 has 4 spectrally distinct peaks, at about 490 nm, about 545 nm, about 580 nm, and about 620 nm, which can be used for energy transfer (FIG. 15). The acceptor fluorophores that can be used include but are not limited to: AlexaFluor 488, AlexaFluor 546, AlexaFluor 647 (FIG. 15), allophycocyanin (APC), and phycoerythrin (PE). Donor and acceptor fluorophores can be conjugated to antibodies using primary amines on antibodies.

The sequence of C-reactive protein (UniProt ID NO. P02741) is shown below:

SEQ ID NO: 2:
MEKLLCFLVLTSLSHAFGQTDMSRKAFVFPKESDTSYVSLKAPLTKPLKAF
TVCLHFYTELSSTRGYSIFSYATKRQDNEILIFWSKDIGYSFTVGGSEILF
EVPEVTVAPVHICTSWESASGIVEFWVDGKPRVRKSLKKGYTVGAEASIIL
GQEQDSFGGNFEGSQSLVGDIGNVNMWDFVLSPDEINTIYLGGPFSPNVLN
WRALKYEVQGEVFTKPQLWP

### Example 4

This example illustrates a method of this disclosure detecting the presence and amounts of ATA IgA and/or ATA IgG in a TR-FRET assay. As shown in FIG. 8A, an isolated tissue transglutaminase protein (tTG) labeled with a donor fluorophore binds to an ATA IgA or IgG labeled with an acceptor fluorophore. The endogenous ATA IgA or IgG analyte is in a sample from a patient (i.e., whole blood sample) and it binds to the isolated tissue transglutaminase protein (tTG) protein labeled with the donor fluorophore, thus, disrupting the FRET signal. In the presence of a high amount of endogenous ATA IgA or IgG analyte, the FRET signal is low, since the endogenous ATA IgA or IgG analyte in the sample (e.g., a whole blood sample) blocks or competes with the binding to the isolated tissue transglutaminase protein (tTG) protein. The decrease in FRET signal is proportional to the level of endogenous ATA IgA or IgG analyte present in the patient's blood as interpolated from a known amount of ATA IgA or IgG calibrators (FIG. 9).

An assay method that can detect, as well as distinguish, the presence or amount of anti-transglutaminase autoantibody (ATA) immunoglobulin A (IgA) and ATA immunoglobulin G (IgG) in a sample is shown in FIG. 8B.

Further, as shown in FIG. 10A, an isolated tissue transglutaminase protein (tTG) labeled with a donor fluorophore binds to an anti-IgA labeled with an acceptor fluorophore in the presence of ATA IgA in a patient's sample. The endogenous ATA IgA analyte is in a sample from a patient (i.e., whole blood sample) and it binds to the isolated tissue transglutaminase protein (tTG) protein labeled with the donor fluorophore and also the anti-IgA labeled with the acceptor fluorophore, thus, bringing the donor and acceptor fluorophores in proximity of each other to generate a FRET signal. In the presence of a high amount of endogenous ATA IgA analyte, the FRET signal is high, since the endogenous ATA IgA analyte in the sample (e.g., a whole blood sample) brings the isolated tissue transglutaminase protein (tTG) protein and the anti-IgA together. The increase in FRET signal is proportional to the level of endogenous ATA IgA analyte present in the patient's blood as interpolated from a known amount of ATA IgA calibrators (FIG. 10B).

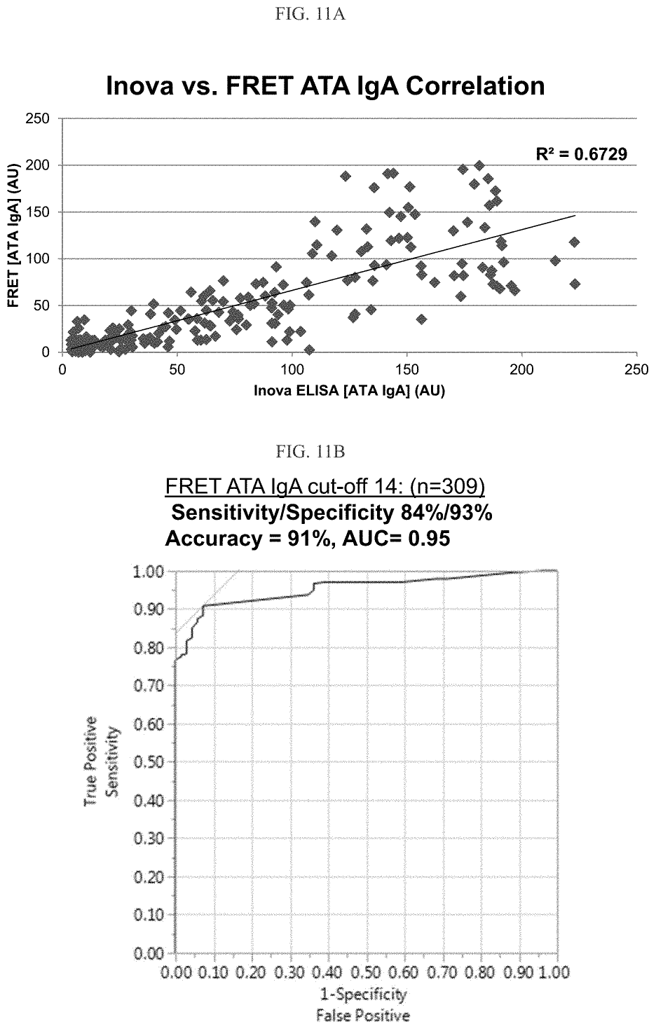

This example illustrates how the method of this disclosure for detecting the presence and amounts of ATA IgA analyte present in a patient sample using a TR-FRET assay in the format shown in FIG. 10A correlates to an ELISA from Inova. For the correlation, the same serum sample was tested using TR-FRET and the Inova ELISA. The results are displayed graphically in FIG. 11A. FIG. 11B illustrates the illustrates the sensitivity and specificity of the present disclosure for ATA IgA using a cut-off of 14 for 309 patient samples.

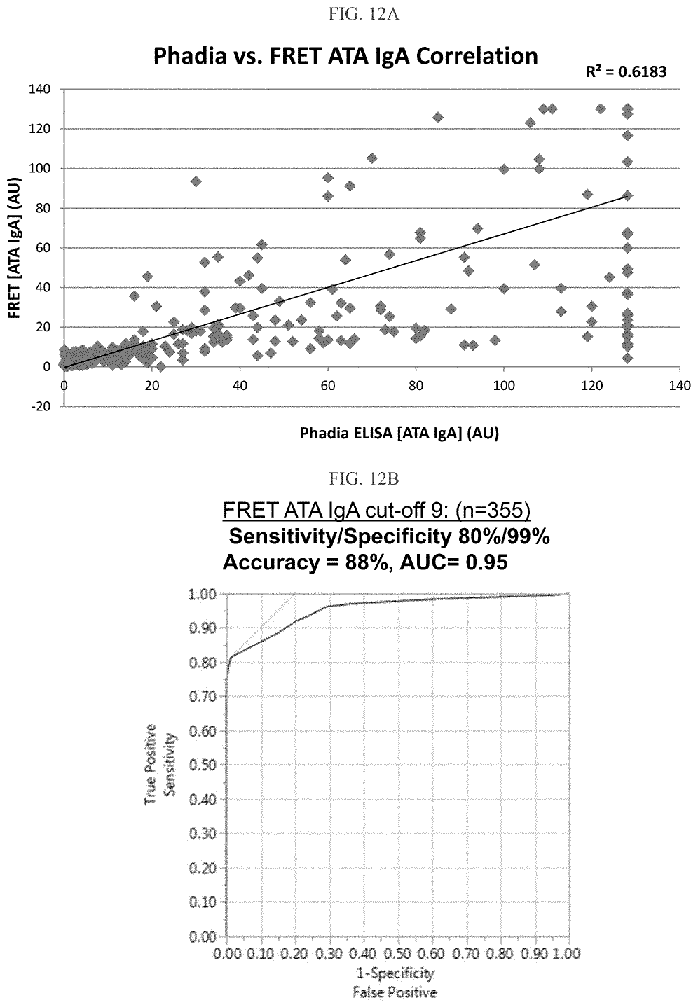

This example illustrates how the method of this disclosure for detecting the presence and amounts of ATA IgA analyte present in a patient sample using a TR-FRET assay in the format shown in FIG. 10A correlates to an ELISA from Phadia. For the correlation, the same serum sample was tested using TR-FRET and the Inova ELISA. The results are displayed graphically in FIG. 12A. FIG. 12B illustrates the illustrates the sensitivity and specificity of the present disclosure for ATA IgA using a cut-off of 9 for 355 patient samples.

Donor fluorophore, Lumi4-Tb (also called Tb-H22TRENIAM-5LIO-NHS, FIG. 13), can be used to label an isolated isolated tissue transglutaminase protein (tTG) protein. Lumi4-Tb has 4 spectrally distinct peaks, at about 490 nm, about 548 nm, about 587 nm, and 621 nm, which can be used for energy transfer (FIG. 15). The acceptor fluorophores that can be used include but are not limited to: AlexaFluor 488, AlexaFluor 546, AlexaFluor 647 (FIG. 15), allophycocyanin (APC), and phycoerythrin (PE). Donor and acceptor fluorophores can be conjugated to antibodies using primary amines on antibodies.

1-21. (canceled)

22. An assay method for detecting the presence or amount of anti-transglutaminase autoantibody (ATA) immunoglobulin A (IgA) and/or ATA immunoglobulin G (IgG) in a sample, the method comprising:
contacting the sample with a complex comprising an anti-tissue transglutaminase antibody labeled with a donor fluorophore and an isolated tissue transglutaminase labeled with an acceptor fluorophore, wherein the anti-tissue transglutaminase antibody comprises a binding epitope to tissue transglutaminase, and wherein the complex emits a fluorescence emission signal associated with fluorescence resonance energy transfer (FRET) when the donor fluorophore is excited using a light source;
incubating the sample with the complex for a time sufficient for ATA IgA and/or ATA IgG in the sample to compete for binding to the isolated tissue transglutaminase labeled with the acceptor fluorophore; and
exciting the sample using a light source to detect a fluorescence emission signal associated with FRET,
wherein an absence of the fluorescence emission signal or a decrease in the fluorescence emission signal relative to the fluorescence emission signal initially emitted by the complex indicates the presence or amount of ATA IgA and/or ATA IgG in the sample.

23. An assay method for detecting the presence or amount of anti-transglutaminase autoantibody (ATA) immunoglobulin A (IgA) and/or ATA immunoglobulin G (IgG) in a sample, the method comprising:
contacting the sample with a complex comprising an anti-tissue transglutaminase antibody labeled with an acceptor fluorophore and an isolated tissue transglutaminase labeled with a donor fluorophore, wherein the anti-tissue transglutaminase antibody comprises a binding epitope to tissue transglutaminase, and wherein the complex emits a fluorescence emission signal associated with fluorescence resonance energy transfer (FRET) when the donor fluorophore is excited using a light source;
incubating the sample with the complex for a time sufficient for ATA IgA and/or ATA IgG in the sample to compete for binding to the isolated tissue transglutaminase labeled with the donor fluorophore; and
exciting the sample using a light source to detect a fluorescence emission signal associated with FRET,
wherein an absence of the fluorescence emission signal or a decrease in the fluorescence emission signal relative to the fluorescence emission signal initially emitted by the complex indicates the presence or amount of ATA IgA and/or ATA IgG in the sample.

24. An assay method for detecting the presence or amount of anti-transglutaminase autoantibody (ATA) immunoglobulin A (IgA) and/or ATA immunoglobulin G (IgG) in a sample, the method comprising:
contacting the sample with a complex comprising an anti-tissue transglutaminase antibody labeled with a donor fluorophore and an isolated tissue transglutaminase labeled with a first acceptor fluorophore, wherein the anti-tissue transglutaminase antibody comprises a binding epitope to tissue transglutaminase, and wherein the complex emits a fluorescence emission signal associated with fluorescence resonance energy transfer (FRET) when the donor fluorophore is excited using a light source;
incubating the sample with the complex for a time sufficient for ATA IgA and/or ATA IgG in the sample to compete for binding to the isolated tissue transglutaminase labeled with the first acceptor fluorophore;
further incubating the sample with an anti-IgA antibody labeled with a second acceptor fluorophore and an anti-IgG antibody labeled with a third acceptor fluorophore; and
exciting the sample using a light source to detect fluorescence emission signals associated with FRET,
wherein a detection of a fluorescence signal emitted by the second acceptor fluorophore indicates the presence or amount of ATA IgA and a detection of a fluorescence signal emitted by the third acceptor fluorophore indicates the presence or amount of ATA IgG in the sample.

25. An assay method for detecting the presence or amount of anti-transglutaminase autoantibody (ATA) immunoglobulin A (IgA) and/or ATA immunoglobulin G (IgG) in a sample, the method comprising:
contacting the sample with a complex comprising an anti-tissue transglutaminase antibody labeled with a first acceptor fluorophore and an isolated tissue transglutaminase labeled with a donor fluorophore, wherein the anti-tissue transglutaminase antibody comprises a binding epitope to tissue transglutaminase, and wherein the complex emits a fluorescence emission signal associated with fluorescence resonance energy transfer (FRET) when the donor fluorophore is excited using a light source;
incubating the sample with the complex for a time sufficient for ATA IgA and/or ATA IgG in the sample to compete for binding to the isolated tissue transglutaminase labeled with the donor fluorophore;
further incubating the sample with an anti-IgA antibody labeled with a second acceptor fluorophore and an anti-IgG antibody labeled with a third acceptor fluorophore; and
exciting the sample using a light source to detect fluorescence emission signals associated with FRET,
wherein a detection of a fluorescence signal emitted by the second acceptor fluorophore indicates the presence or amount of ATA IgA and a detection of a fluorescence signal emitted by the third acceptor fluorophore indicates the presence or amount of ATA IgG in the sample.

26. The method of claim 24, further comprising, prior to the further incubating step, determining the total amount of ATA IgA and IgG by exciting the sample using a light source to detect a fluorescence emission signal associated with FRET, wherein an absence of the fluorescence emission signal or a decrease in the fluorescence emission signal relative to the fluorescence emission signal initially emitted by the complex indicates the total amount of ATA IgA and ATA IgG in the sample.

27. The method according to claim 22, wherein the concentration of ATA IgA in the blood is about 7 mg/dL to about 4,000 mg/dL.

28. The method according to claim 27, wherein a normal concentration of ATA IgA in the blood is about 70 mg/dL to about 400 mg/dL.

29. The method according to claim 27, wherein an elevated concentration of ATA IgA in the blood is at least above 400 mg/dL.

30. (canceled)

31. The method according to claim 22, wherein the concentration of ATA IgG in the blood is about 20 mg/dL to about 4,000 mg/dL.

32. The method according to claim 31, wherein a normal concentration of ATA IgG in the blood is about 200 mg/dL to about 400 mg/dL.

33. The method according to claim 31, wherein an elevated concentration of ATA IgG in the blood is at least above 400 mg/dL.

34. The method according to claim 33, wherein an elevated concentration of ATA IgG in the blood is at least above 800 mg/dL.

35. The method according to claim 22, wherein the FRET emission signals are time resolved FRET emission signals.

36. The method according to claim 22, wherein the sample is a biological sample.

37. The method according to claim 36, wherein the biological sample is selected from the group consisting of whole blood, urine, a fecal specimen, plasma, and serum.

38. The method according to claim 37, wherein the biological sample is whole blood.

39. The method according to claim 22, wherein the donor fluorophore is a terbium cryptate.

40. The method according to claim 22, wherein the acceptor fluorophore is selected from the group consisting of fluorescein-like (green zone), Cy5, DY-647, Alexa Fluor 488, Alexa Fluor 546, Alexa Fluor 647, allophycocyanin (APC), and phycoerythrin (PE).

41. The method according to claim 22, wherein the light source provides an excitation wavelength between about 300 nm to about 400 nm.

42. The method according to claim 22, wherein the fluorescence emission signals emit emission wavelengths that are between about 450 nm to about 700 nm.
